(19) United States
(12) Patent Application Publication
(10) Pub. No.: US 2025/0281495 A1
(43) Pub. Date: Sep. 11, 2025

(54) METHODS FOR INHIBITING INFLAMMATION AND PROGRESSION OF ATHEROSCLEROTIC PLAQUES AND CARDIOVASCULAR EVENTS IN PATIENTS WITH CEREBRO- AND CARDIO-VASCULAR DISEASE

(71) Applicant: ICAHN SCHOOL OF MEDICINE AT MOUNT SINAI, New York, NY (US)

(72) Inventor: Chiara Giannarelli, New York, NY (US)

(73) Assignee: ICAHN SCHOOL OF MEDICINE AT MOUNT SINAI, New York, NY (US)

(21) Appl. No.: 18/278,526

(22) PCT Filed: Feb. 24, 2022
(86) PCT No.: PCT/US22/17777

§ 371 Date: Aug. 23, 2023

Related U.S. Application Data
(60) Provisional application No. 63/153,645, filed on Feb. 25, 2021.

(51) Int. Cl. A61K 31/5377; A61K 31/397; A61K 31/404; A61K 31/453; A61K 31/506; A61K 31/517; A61K 45/06; A61P 9/10

(52) U.S. Cl. CPC A61K 31/5377; A61K 31/397; A61K 31/404; A61K 31/453; A61K 31/506; A61K 31/517; A61K 45/06; A61P 9/10

(57) ABSTRACT

The present disclosure provides methods for treating cerebrovascular or cardiovascular disease by inhibiting or reducing inflammation in patients using known compounds, including saracatinib.

### RELATED APPLICATION

This application is a § 371 national stage of PCT International Application No. PCT/US22/17777, filed Feb. 24, 2022, claiming the benefit of U.S. Provisional Application No. 63/153,645 filed Feb. 25, 2021, which is incorporated herein by reference in its entirety.

### BACKGROUND

Despite lifestyle improvements and routine use of statins, atherosclerotic cardiovascular (CV) disease (CVD), including myocardial infarction and stroke, is still the leading cause of death worldwide with an estimated 18 million deaths attributed to CV disease in 2016. Inflammation and plaque instability are thought to be a major pathophysiological driver of disease even in patients with well-controlled LDL levels.

Chronic inflammation promotes atherogenesis independently of hyperlipidemia. New drugs other than lipid lowering compounds are urgently needed to treat atherosclerotic CVD. This is particularly true after the repeated failure of phase-2 clinical trials testing new anti-inflammatory drugs identified by traditional single-target drug discovery methods. New therapeutics are urgently needed to treat chronic inflammation that still exists even in patients optimally treated with lipid-lowering agents such as statins.

Inflammation is intimately involved at all stages of atherosclerosis and interventions targeting inflammation have become an attractive strategy to reduce this residual cardiovascular risk. The Canakinumab Antiinflammatory Thrombosis Outcome Study (CANTOS) first, and the Colchicine Cardiovascular Outcomes Trial (COLCOT) next, proved for the first time that targeting inflammation effectively reduces the risk for secondary cardiovascular events in patients. The initial enthusiasm from these studies was however diminished by the negative results of the CIRT trial. In this study, low-dose methotrexate—the gold standard therapy for rheumatic arthritis—did not reduce either markers of inflammation or cardiovascular events in patients with previous acute myocardial infarction or multivessel coronary disease who had either type 2 diabetes or the metabolic syndrome. The lesson learned from the CIRT is that targeting inflammation broadly is not an effective strategy to reduce cardiovascular risk in patients with atherosclerosis cardiovascular disease.

A one-size-fits all immunotherapeutic approach has also proven ineffective due to variation in patient responses. A sub-analysis of the CANTOS trial showed a greater benefit with canakinumab, a monoclonal antibody against IL-1β, in those patients whose levels of the inflammatory biomarker high-sensitivity C-reactive protein decreased below 2 mg/L at three months into treatments. Other clinical trials showed that effective immunotherapies need to be tailored to specific groups of patients. For example, low-dose colchicine reduced composite cardiovascular outcomes (myocardial infarction or ischemia-driven revascularization) in patients with stable coronary artery disease in the LoDoCo2 trial, but the incidences of death from any cause and non-cardiovascular death were higher in the colchicine-treated group than in the placebo group. Moreover, in the COPS trial patients with acute coronary syndrome that received colchicine had higher mortality and no benefit in reducing cardiovascular outcomes at 12 months compared with placebo.

The traditional drug discovery approaches may have contributed to the lack of significant conceptual advances in the field, basing most candidate anti-inflammatory treatments on single targets or epidemiological evidence of cardiovascular benefits of existing immunotherapies in patients treated for conditions other than cardiovascular disease like rheumatic arthritis. Other treatments stem from mouse models, an approach that poses intrinsic translational limitations due to possible discrepant immune responses to disease state across species. These drawbacks can be easily overcome by studying disease specific immune alterations directly in humans. However, until very recently, the constrained access to human tissue samples and lack of innovative high-dimensional technologies to examine them, have limited most research effort to the analysis of individual cell populations and few markers.

Therefore, there is a need in the art for new methods to identify targets for treating CVD and identifying therapeutic approaches to reduce inflammation and residual risk of cardiac death.

### SUMMARY

The present disclosure overcomes the deficiencies noted above by providing methods for treating cardiovascular disease and cerebrovascular disease using compounds that reduce inflammation associated with cardiovascular disease.

The immune system is a hierarchical set of molecular and cellular networks that govern the immune interactions that lead to cardiovascular disease (CVD). This disclosure provides for the first time a novel combination of systems biology and computational drug repurposing analyses to identify target compounds with the ability to restore the function of immune networks relevant to human atherosclerosis at the single cell level.

The present disclosure integrates single-cell CyTOF mass cytometry and gene expression analysis of human immune cell perturbations with a computational drug repurposing method that identified new anti-inflammatory and anti-atherosclerotic properties for existing compounds. CyTOF screening showed that the dual-specific inhibitor of Src and Abl, protein tyrosine kinases saracatinib (AZD-0530) and Ro 31-8220 mesylate reversed the input inflammatory signature identified in patients. Saracatinib was next validated in a mouse model of atherosclerosis progression and in a large rabbit model of atherosclerosis using molecular imaging and histology.

Using the disclosed methods, the inventors discovered new candidate anti-inflammatory drugs for treating CVD—including, for example, Ro 31-8220 mesylate, Alvocidib; AZD8055; Saracatinib; and PF-562271 HCl. The inventors discovered that saracatinib inhibited the phosphorylation of key intracellular kinases (i.e., CREB and S6) activated in specific cell types (e.g., monocytes and DCs) by plasma of patients with carotid atherosclerosis.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 shows the study design used to identify molecules for treating CVD.

FIGS. 2A-2B show multiplexed mass cytometry and reveals signaling functions that mark immune response to plasma of atherosclerotic patients. FIG. 2A. Heatmap summary of CyTOF mass cytometry data, ordered by stimulatory plasma condition and similarity for all cell type-phosphoprotein pairs. FIG. 2B. PBMCs were visualized using viSNE, and major immune subsets were defined based on canonical expression patterns. Single cell signaling patterns were visualized across this immune map in response to healthy donor or atherosclerotic plasma.

FIGS. 3A-3C show multiplexed mass cytometry and reveals signaling functions that mark immune response to plasma of atherosclerotic patients. FIG. 3A. Heatmap summary of CyTOF mass cytometry data, ordered by stimulatory plasma condition and similarity of cell type-phosphoprotein pairs. FIG. 3B. Magnified view of the most plasma sensitive cell types, CD14+ and CD16+ monocytes and CD1c+ dendritic cells, highlights the activation of specific intracellular markers in each cell type in response to atherosclerosis plasma. FIG. 3C. Subset of significant signaling responses is visualized as viSNE plots, in which each cell is colored according to the intensity of the indicated intracellular signaling response. Phosphorylation responses in each cell type can be mapped using the viSNE dot plot, which is colored by cell identity on top.

FIGS. 4A-4D show changes in gene expression profile of healthy PBMCs stimulated with atherosclerotic plasma. Principal component analysis (PCA) of RNA-seq (FIG. 4A) and corresponding CyTOF mass cytometry data (FIG. 4B). FIG. 4C. Heat map of DEGs with the largest variance based on DESeq analysis (size factor normalized) showing transcripts with absolute log₂ fold change>1.2 and normalized sequence counts>4. (FIG. 4D). Log₂ fold-change distribution of 4823 differentially expressed genes (DEGs) in response to atherosclerotic vs. healthy donor plasma. Significant DEGs (DESeq, Benjamini-Hochberg, p<0.05) are highlighted in red. Enriched GO biological processes of the significant up-and down-regulated genes are shown on the right and ordered based on Enrichr's rank-based statistic. Pathways analysis (left panel).

FIGS. 5A-5C show luminex analysis of cytokines expressed by PBMCS stimulated with atherosclerotic (red) vs healthy plasma (blue).

FIG. 6 shows filtered cross-correlations of RNA-seq and aggregate mass cytometry data. Enriched GO terms from gene expression data. Hierarchically ordered heat map of Pearson's correlations between gene expression and phosphoprotein-cell type pairs. Only DEGs with GO functional enrichment included. Pairs of phosphoprotein and cell-type with the highest median cross-correlation with RNA-seq data. ChiP-seq libraries analysis.

FIG. 7 shows small molecules that are predicted to reverse the input inflammatory signatures seen in PBMCs that were stimulated with atherosclerotic plasma.

FIG. 8 shows CyTOF mass-cytometry measurement of the specific inhibition of kinase phosphorylation (i.e., pCREB, pp38, pMAPKA2P, pS6, pERK1/2) in each cell type at the single cell level for each drug.

FIG. 9 shows CyTOF mass-cytometry measurement of the specific inhibition of kinase phosphorylation (i.e., pCREB, pp38, pMAPKA2P, pS6, pERK1/2) in each cell type at the single cell level for each drug.

FIGS. 10A-10D show multiplexed mass cytometry reveals the effect of candidate small molecules on signaling dynamics induced by atherosclerotic plasma. (FIG. 10A). Bulk PBMCs signaling response to atherosclerotic plasma alone (plasma) and in combination with candidate small molecules (1-6). Single-cell phosphorylation was measured by CyTOF, aggregated by the population-wide median, and standardized across biological repeats. (FIG. 10B). Phosphorylation responses in cell types CD1c+DCs, CD14+ and CD16+ monocytes, showing additional details of cell-type-specific signaling and more specific clustering of biological repeats. (FIG. 10C). Statistics of cell-type-specific phosphorylation with positive values indicating up-regulation and negative values down-regulation. Plasma response is compared to no plasma treatment. Each small molecule combined with plasma treatment is compared to plasma treatment alone. Points outside the grey box indicate significance (p<0.05, df=6). (FIG. 10D). viSNE plots highlight the effect of Saracatinib (drug 4) and Mevastatin (drug 6) on CREB phosphorylation induced by atherosclerotic plasma.

FIG. 11 shows CyTOF testing of saracatinib for its efficacy to inhibit the phosphorylation of intracellular kinases in PBMCs of patients with carotid (CEA) and coronary artery disease (CAD) incubated with autologous vs. healthy donor plasma.

FIG. 12 shows the optimal 50% inhibitory concentration (EC50) of the compound as determined by CyTOF.

FIG. 13 shows measurements of Plaque burden using Image Pro plus. For analysis of plaque composition (i.e., infiltration of inflammatory cell), the upper part of the heart and aorta was excised from mice of each group and embedded in OCT to generate aortic root sections for immunohistochemistry with antibodies against CD68 (monocyte/macrophages) and CD45 (pan-leukocytes).

FIG. 14 shows RNA sequencing analysis of atherosclerotic aortas from mice fed with: western diet (HFD); HFD+saracatinib (6.25-25 mg/kg/d); HFD+Atorvastatin (10 mg/kg/d); or HFD+saracatinib (6.25-25 mg/kg/d)+Atorvastatin (10 mg/kg/d), which revealed the upregulation of genes involved in fatty acid oxidation, TCA cycles and PPAR signaling in the saracatinib treated group. These pathways are upregulated in M2 proresolving macrophages that drive plaque regression. Fox-p3 a marker of Tregs, also associated with regression, was also upregulated.

FIG. 15 shows the analysis of a rabbit model of atherosclerosis that develops complex atherosclerotic lesions. ¹⁸F-FDG-uptake was used as a marker of plaque macrophage content (which correlates with plaque macrophage content in both rabbits and patients). ¹⁸F fluorodeoxyglucose (FDG) positron emission topography (PET) is used to quantify plaque inflammation combined with magnetic resonance imaging (MRI) to quantify plaque size. Image analysis uses Standardized Uptake Value (SUV) as a measure of ¹⁸F fluorodeoxyglucose (FDG) positron emission tomography (PET). The vessel wall area MRI is used to quantify plaque size. The results show that ¹⁸F-FDG-uptake was reduced by saracatinib alone or in combination with atorvastatin, and histological analysis of rabbit plaques showed that plaque inflammation was reduced (as indicated by the PET results) and lipid content was also reduced.

FIG. 16 shows the effect of 5 drugs on the expression of cytokines following stimulation of PBMCs with atherosclerotic plasma. The figure reveals a heat map of cytokine expression following treatment with Drug 1, 2, 3, 4, 5 or vehicle alone in PBMCs stimulated with atherosclerotic plasma.

FIGS. 17A-17E provide a Pint Point plot showing the effect of Drugs 1-5 vs. Vehicle on PBMCs stimulated with atherosclerotic plasma. The grey scaling designates fold change, and significance is determined by the size of the circle (bigger=more significant).

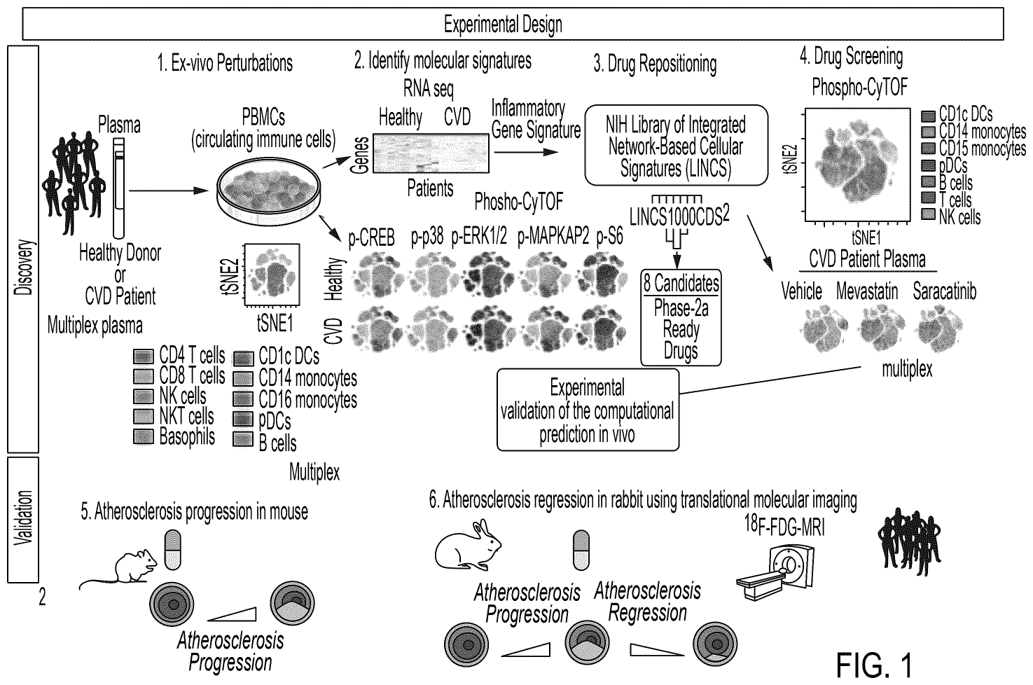

### DETAILED DESCRIPTON

The present disclosure provides methods for treating cerebrovascular or cardiovascular disease in a subject in need thereof by administering a compound selected from: Ro 31-8220 mesylate; Alvocidib; AZD8055; Saracatinib; and PF-562271 HCl.

The present disclosure provides methods for treating cardiovascular disease in a subject in need thereof by administering saracatinib.

The present disclosure provides methods for treating cardiovascular disease in a subject in need thereof by administering Ro 31-8220 mesylate.

The present disclosure provides methods for treating cardiovascular disease in a subject in need thereof by administering Alvocidib.

The present disclosure provides methods for treating cardiovascular disease in a subject by administering AZD8055.

The present disclosure provides methods for treating cardiovascular disease in a subject in need thereof by administering PF-562271 HCl.

The present disclosure provides methods for treating cardiovascular disease in a subject in need thereof by administering a compound selected from: Ro 31-8220 mesylate; Alvocidib; AZD8055; Saracatinib; and PF-562271 HCl, wherein the treating also includes providing to the subject a statin, a PCSK9 inhibitor, or ezetimibe.

The present disclosure provides methods for reducing inflammation associated with cardiovascular disease in a subject in need thereof by administering a compound selected from: Ro 31-8220 mesylate; Alvocidib; AZD8055; Saracatinib; and PF-562271 HCl.

The present disclosure provides methods for reducing inflammation associated with cardiovascular disease in a subject in need thereof by administering saracatinib.

The present disclosure provides methods for reducing inflammation associated with cardiovascular disease in a subject in need thereof by administering Ro 31-8220 mesylate.

The present disclosure provides methods for reducing inflammation associated with cardiovascular disease in a subject in need thereof by administering Alvocidib.

The present disclosure provides methods for reducing inflammation associated with cardiovascular disease in a subject in need thereof by administering AZD8055.

The present disclosure provides methods for reducing inflammation associated with cardiovascular disease in a subject in need thereof by administering PF-562271 HCl.

The present disclosure provides methods for reducing inflammation associated with cardiovascular disease in a subject in need thereof by administering a compound selected from: Ro 31-8220 mesylate; Alvocidib; AZD8055; Saracatinib; and PF-562271 HCl, wherein the treating also includes providing to the subject a statin, a PCSK9 inhibitor, or ezetimibe.

### Definitions

Unless defined otherwise, all technical and scientific terms used herein have the meaning commonly understood by a person skilled in the art to which this disclosure belongs. The meaning and scope of the terms should be clear, however, in the event of any latent ambiguity, definitions provided herein take precedent over any dictionary or extrinsic definition.

As used herein, the following terms have the meanings ascribed to them below, unless specified otherwise. The terminology used herein is for the purpose of describing particular embodiments only and is not intended to be limiting of the disclosure. As used herein, the singular forms “a”, “an” and “the” are intended to include the plural forms as well, unless the context clearly indicates otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “effective amount” or “therapeutically effective amount” refers to a quantity of a drug sufficient to achieve a desired effect or a desired therapeutic effect. In the context of therapeutic applications, the amount of the drug administered to the subject can depend on the type and severity of the disease or symptom and on the characteristics of the individual, such as general health, age, sex, body weight and tolerance to drugs. The skilled artisan will be able to determine appropriate dosages depending on these and other factors.

As used herein, the terms “treat,” “treatment,” or “treating” includes any treatment of a condition or disease in a subject, or particularly a human, and may include: (i) preventing the disease or condition from occurring in the subject which may be predisposed to the disease but has not yet been diagnosed as having it; (ii) inhibiting the disease or condition, i.e., arresting or slowing down its progression; relieving the disease or condition, i.e., causing regression of the condition; or (iii) ameliorating or relieving the conditions caused by the disease, i.e., symptoms of the disease. “Treat,” “treatment,” or “treating,” as used herein, could be used in combination with other standard therapies or alone.

As understood by a person of skill in the art, cardiovascular disease is defined as atherosclerotic cardiovascular disease that is due to atherosclerotic plaque formation. Plaques may partially or totally block blood flow through large- or medium-sized arteries in the heart, brain, pelvis, legs, arms, or kidneys. This can lead to conditions such as: coronary heart disease (plaque in arteries in or leading to the heart) and Carotid artery disease (plaque in neck arteries supplying blood to the brain).

### EXAMPLES

In order that the invention described herein may be more fully understood, the following examples are set forth. The examples described in this application are offered to illustrate the methods, compositions, and systems provided herein and are not to be construed in any way as limiting their scope.

### Example 1

### Phospho-CyTOF Identifies Systemic Immune Dysregulations in PBMCs of Atherosclerotic Patients

To identify systemic immune dysregulations in atherosclerosis patients, the inventors developed a multiscale system approach employing phospho-CyTOF to comprehensively characterize the activation state of immune populations with single-cell resolution. The inventors exposed PBMCs isolated from patients with carotid or coronary atherosclerosis (n=9) either to autologous plasma or plasma from healthy donors (n=5) and interrogated the activation of major immune signaling pathways across all major immune subsets using a panel of antibodies against 18 surface markers and 10 intracellular phospho-proteins.

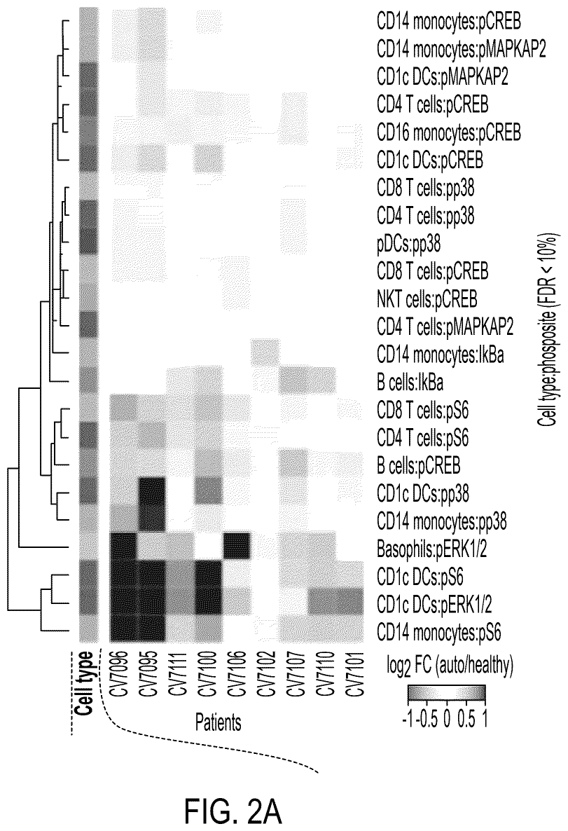

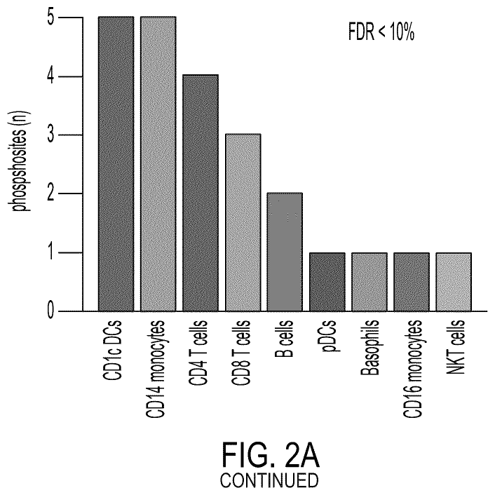

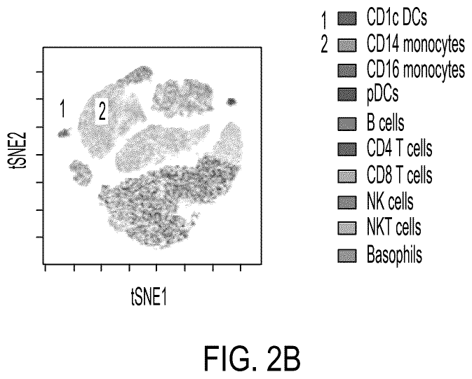

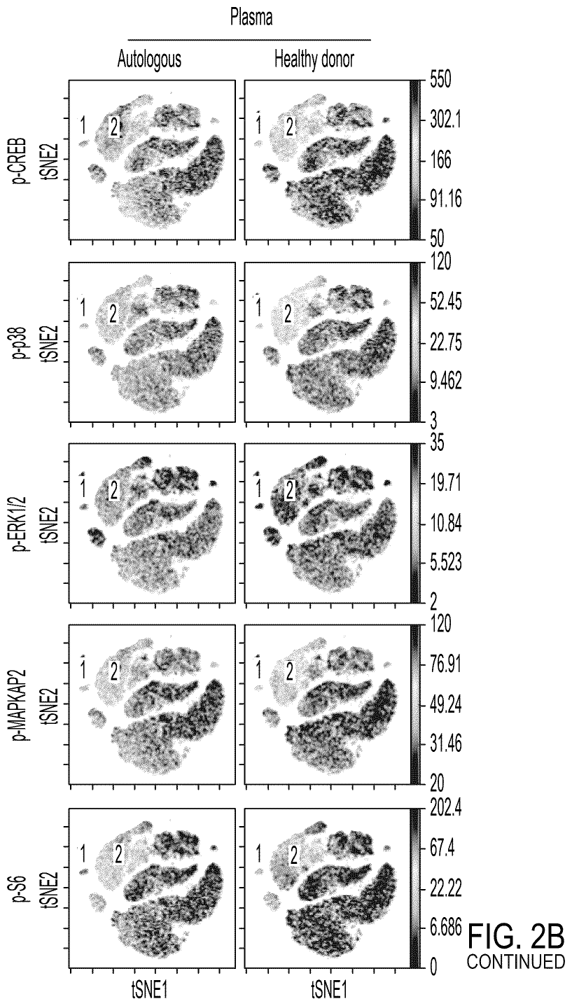

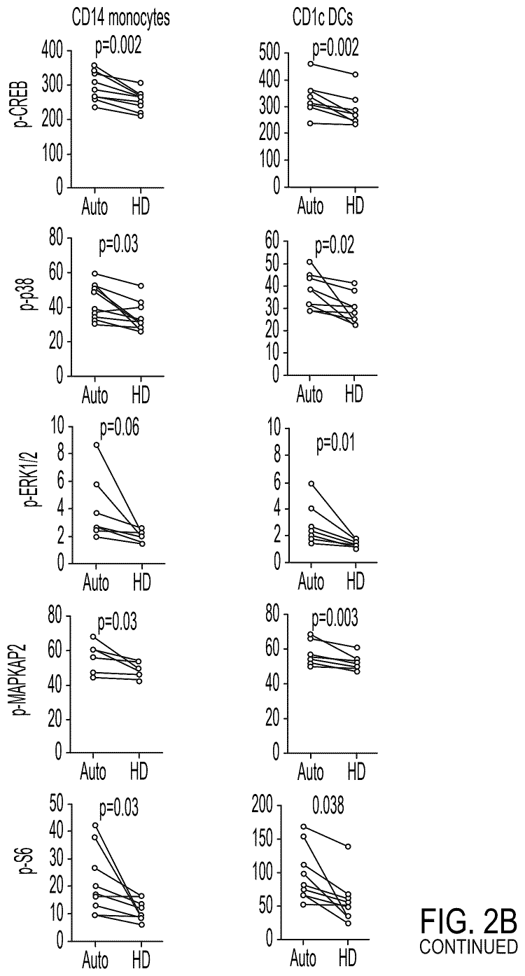

First, the inventors applied viSNE to visualize and define 10 major immune cell populations (B cells, Basophils, CD1c DCs, CD4 T cells, CD8 T cells, CD14 and CD16 monocytes, NK cells, NK T cells, and pDCs) based on canonical marker expression patterns. The inventors then evaluated the relative level of phosphorylation of 10 proteins (IkBa, CREB, ERK1/2, MAPKAP2, p38, PLCg2, S6, STAT1, STAT3, and STAT5) across this immunological map. This analysis allowed the visualization of functional pathways induced in response to plasma from atherosclerosis patients vs. healthy donors. Next, data were integrated to derive 100 cell type-phosphoprotein pairs that were compared across all the different plasma treatments, revealing that CD14+ monocytes and CD1c+ DCs exhibited the greatest immune activation, reflected by the increased phosphorylation of CREB, p38, ERK1/2, MAPKAP2 and S6 in response to autologous atherosclerotic plasma vs. healthy plasma (FIG. 2). These results reveal that plasma of patients with atherosclerotic disease drives specific innate immune cell signaling functions that mark the inflammatory response in atherosclerotic disease.

### Example 2

### Atherosclerotic Plasma Drives Innate Immune Activation and Inflammatory Transcriptional Reprogramming

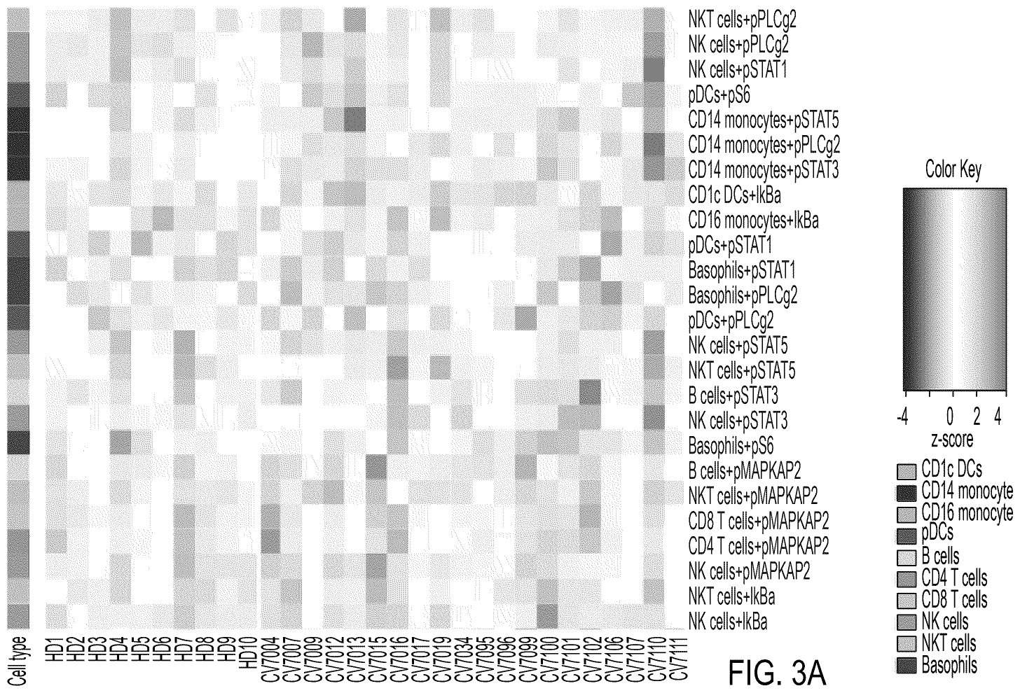

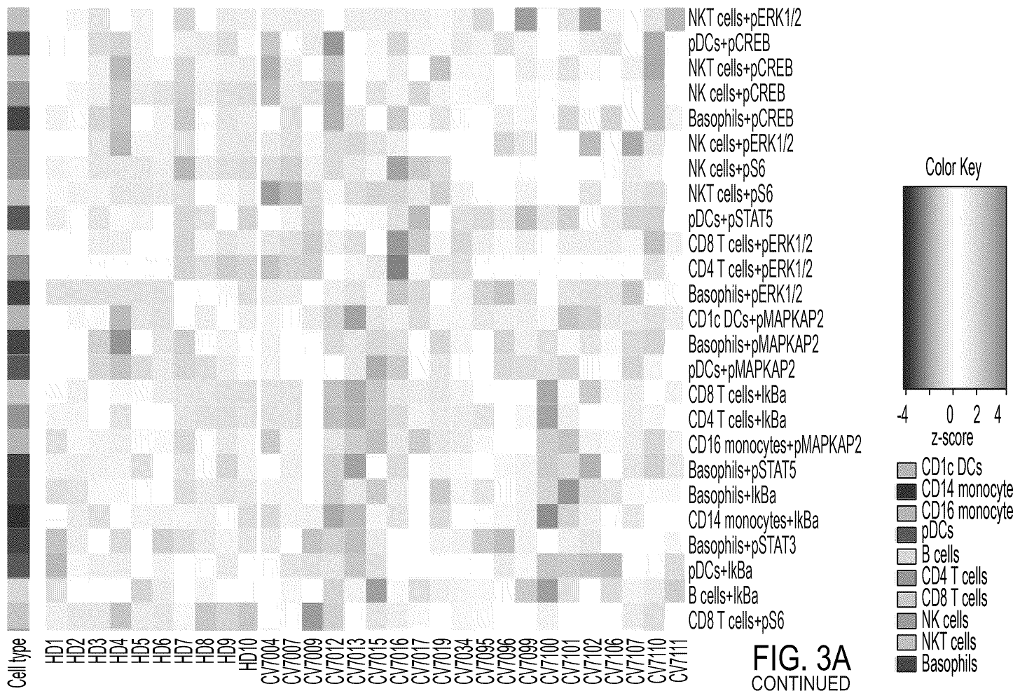

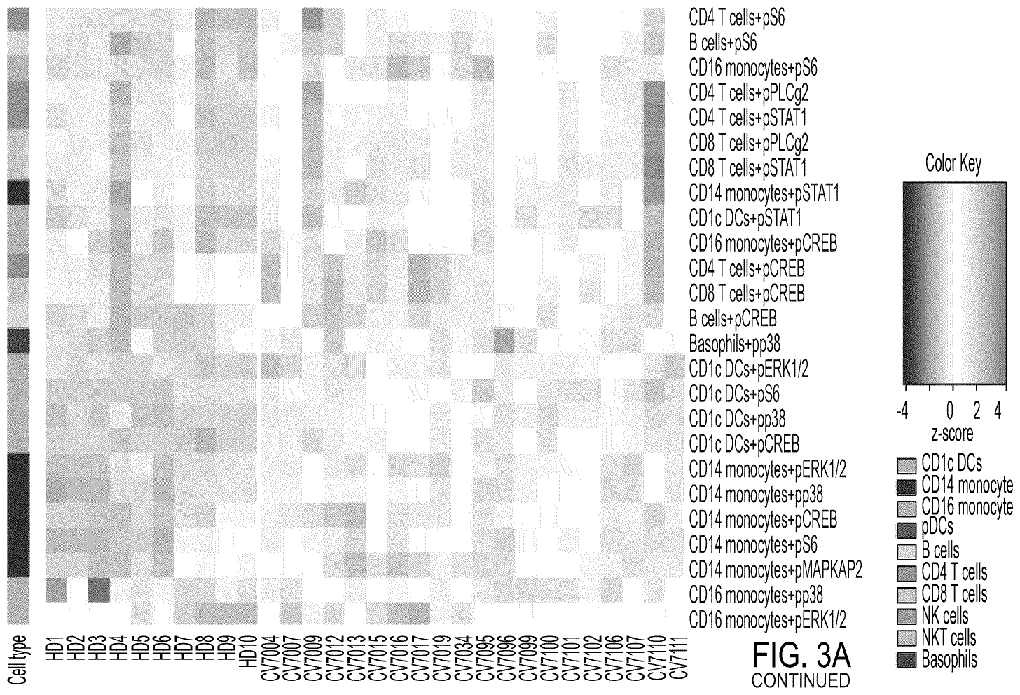

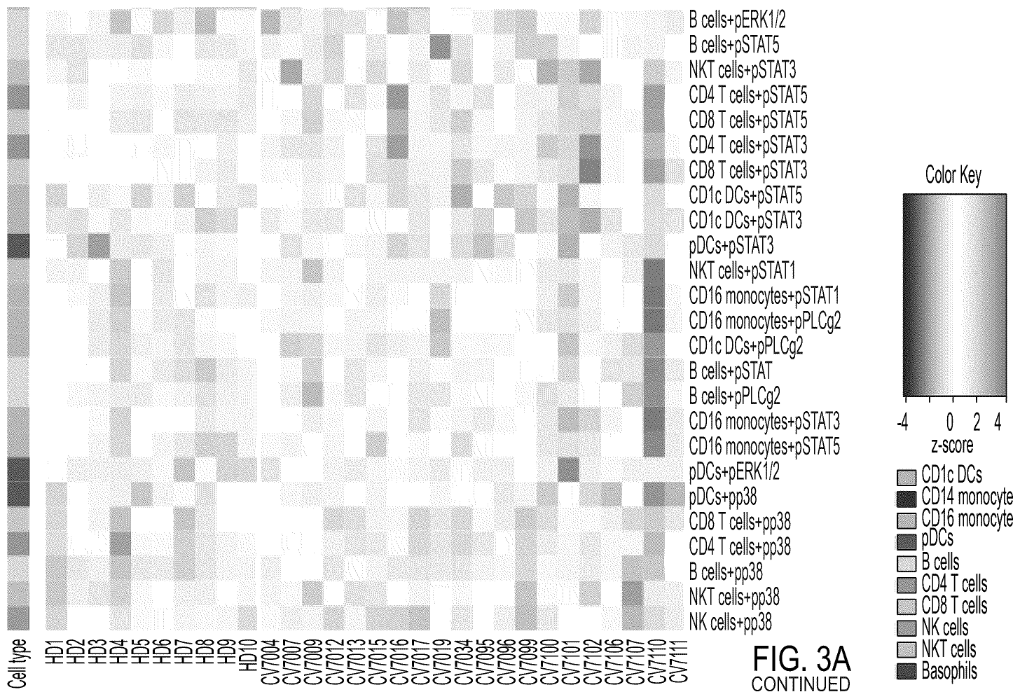

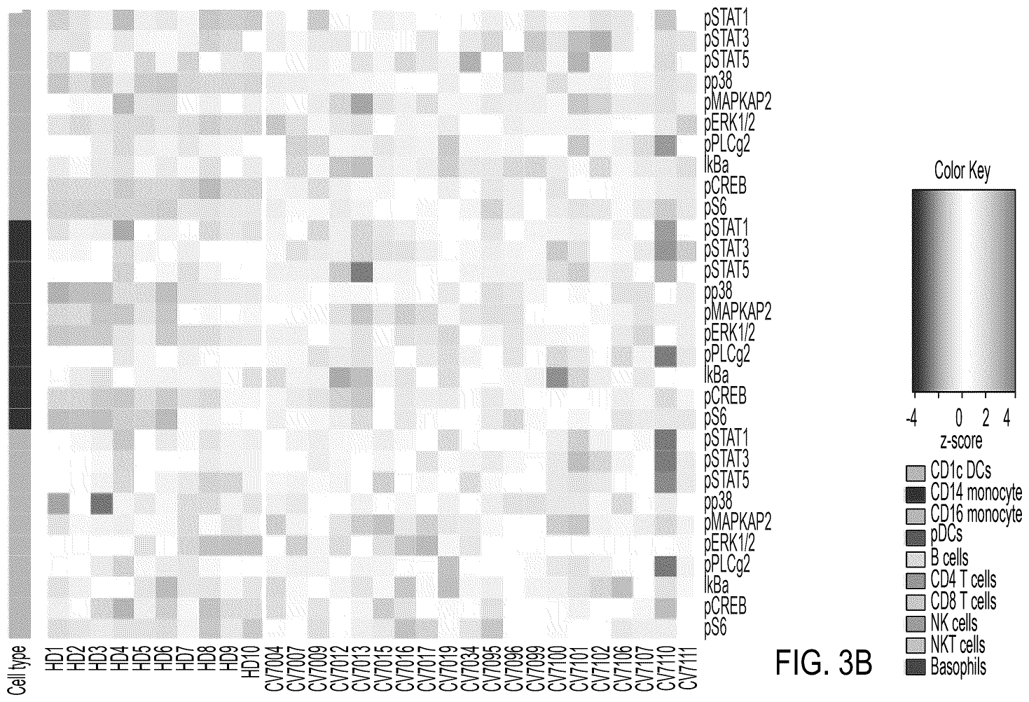

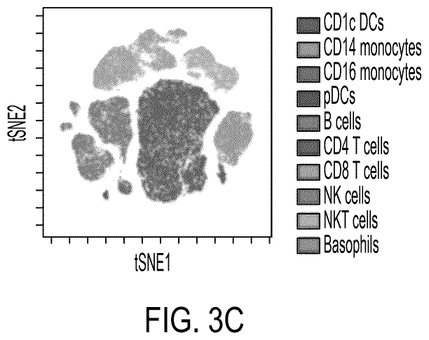

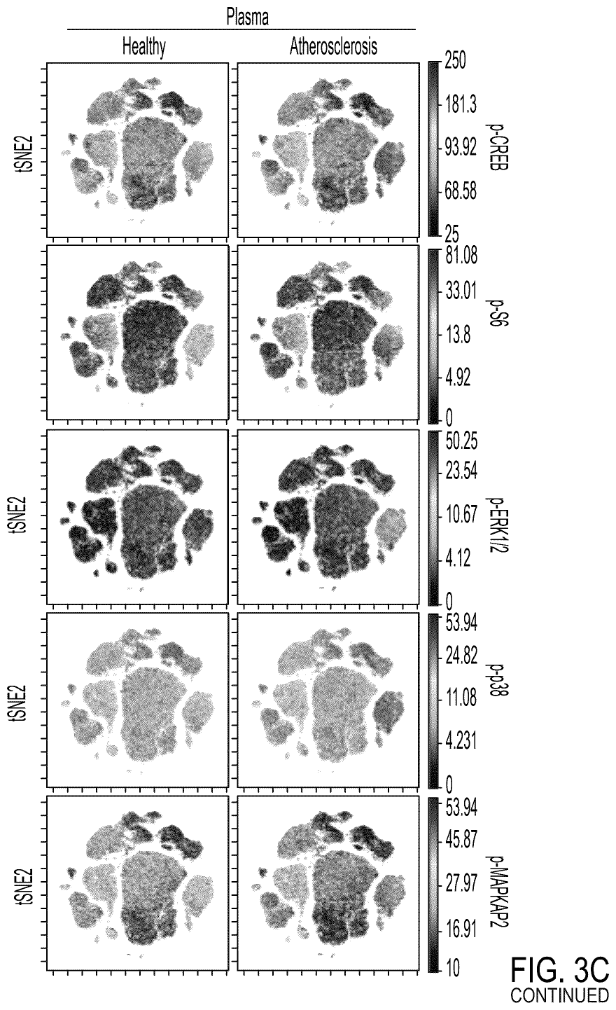

To determine whether immune responses were entirely dependent on atherosclerotic plasma or due to intrinsic cellular features of patients' PBMCs, the inventors exposed PBMCs isolated from healthy individuals to either plasma of patients with atherosclerosis (n=20) or healthy donors (n=10) and interrogated the activation of the same signaling pathways across all major immune subsets using the same panel of 18 surface markers and 10 intracellular phosphor-proteins (FIG. 3). Using a consistent analysis strategy, the inventors applied viSNE to identify and visualize the same major immune cell populations based on canonical marker expression patterns, and then visualized the relative expression of the same 10 phospho-proteins across this immunological map. This approach demonstrated that exposure of healthy PBMCs to atherosclerotic patient plasma largely recapitulated the elevated immune activation signature that was observed in patients' PBMCs. Monocytes and CD1c⁺ DCs exhibited the greatest immune activation, showing significantly elevated phosphorylation of CREB, p38, MAPKAP2, ERK1/2 and S6 in response to atherosclerotic plasma. These results indicate that plasma of patients with atherosclerotic disease drives specific innate immune cell signaling functions that comprise a systemic inflammatory signature in atherosclerotic disease shaped by the interaction of circulating immune cells with plasma factors.

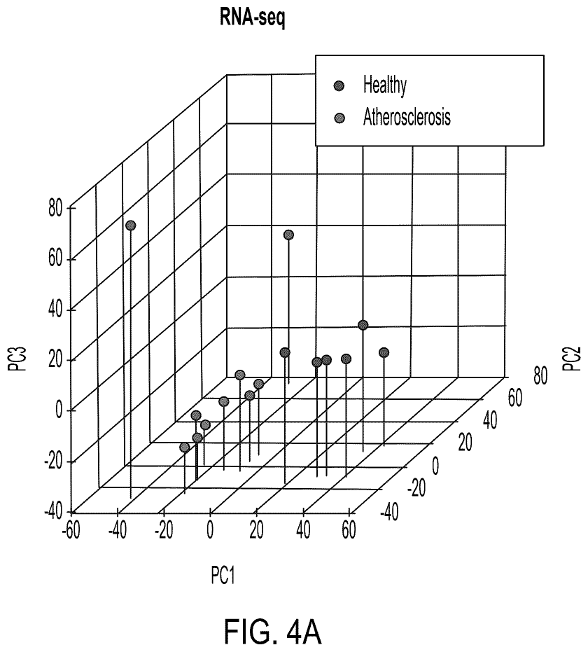

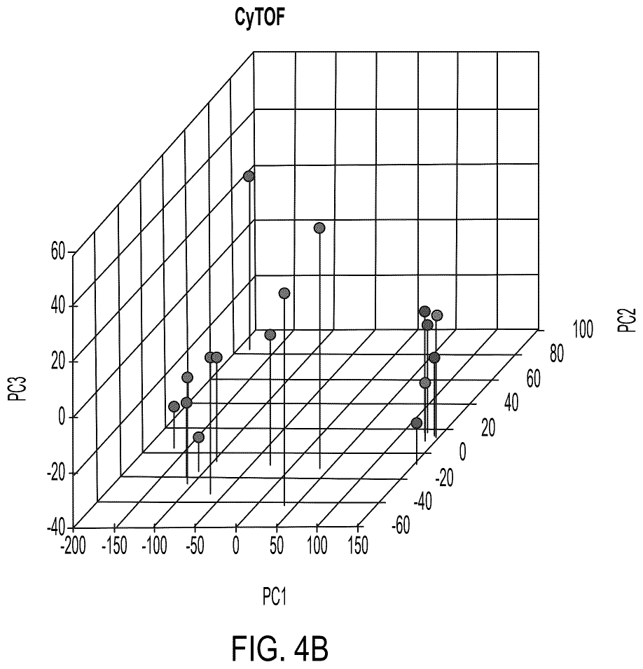

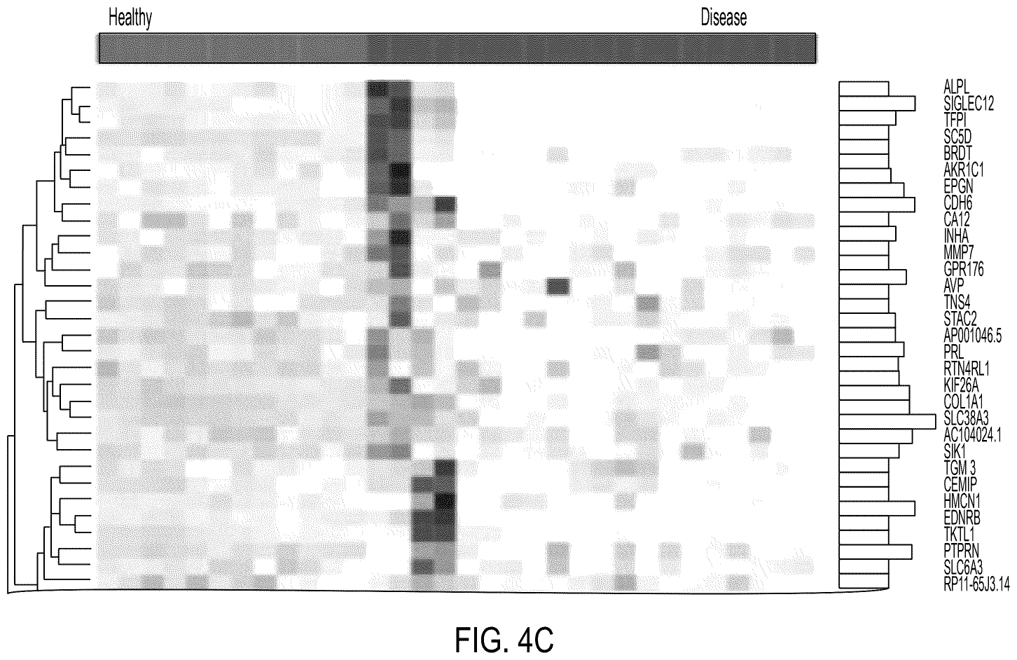

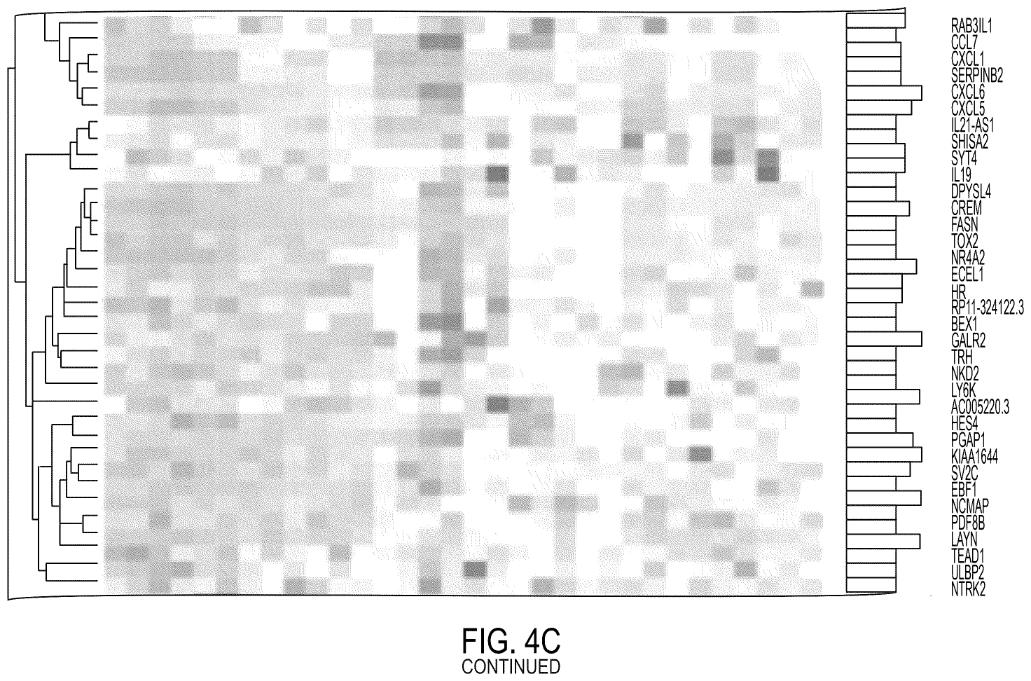

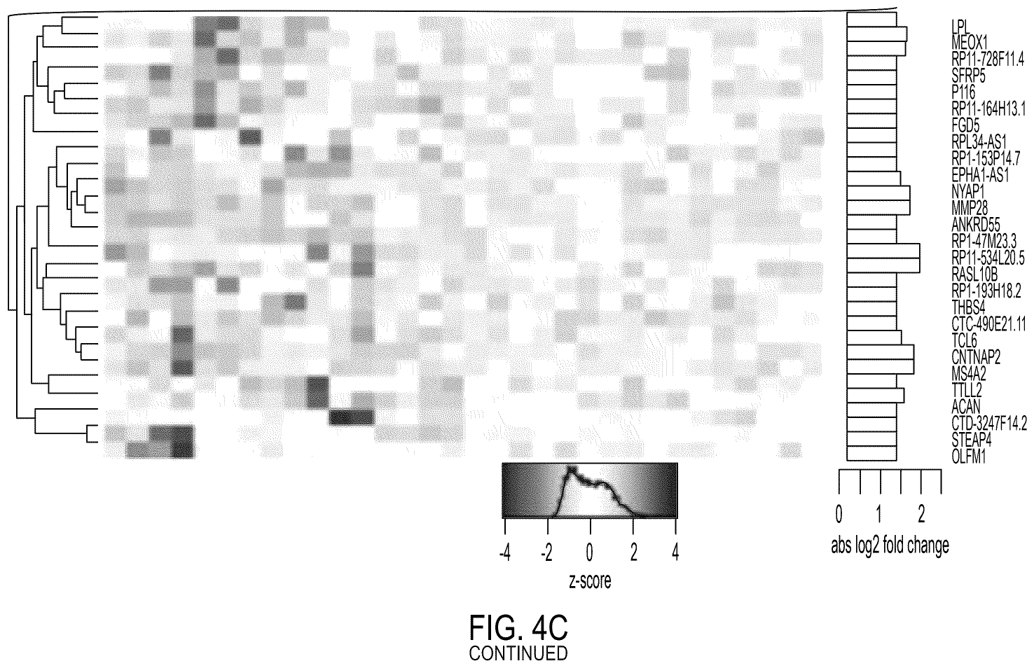

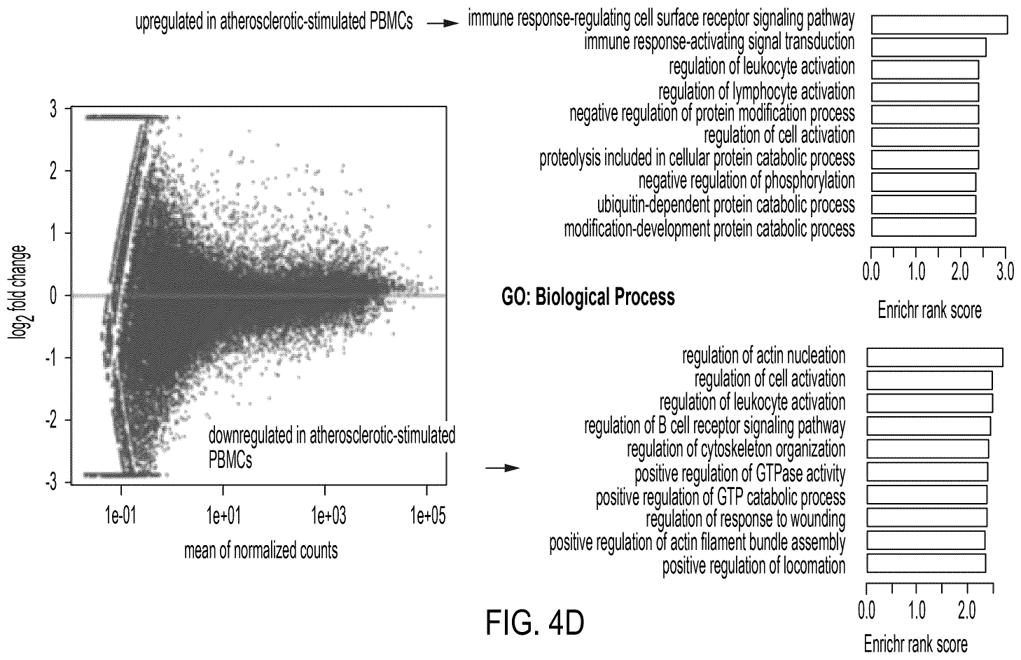

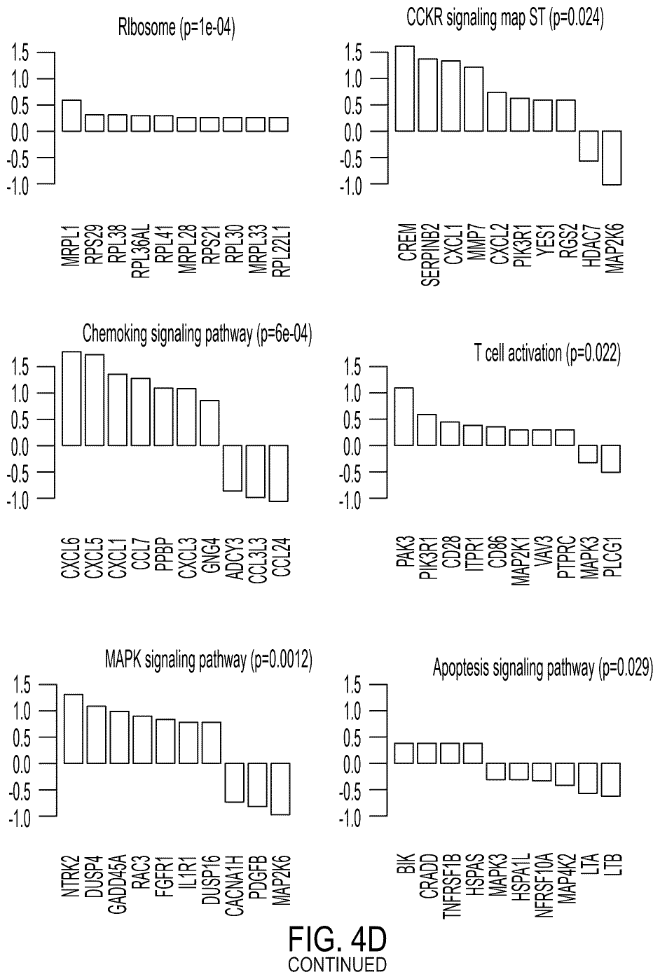

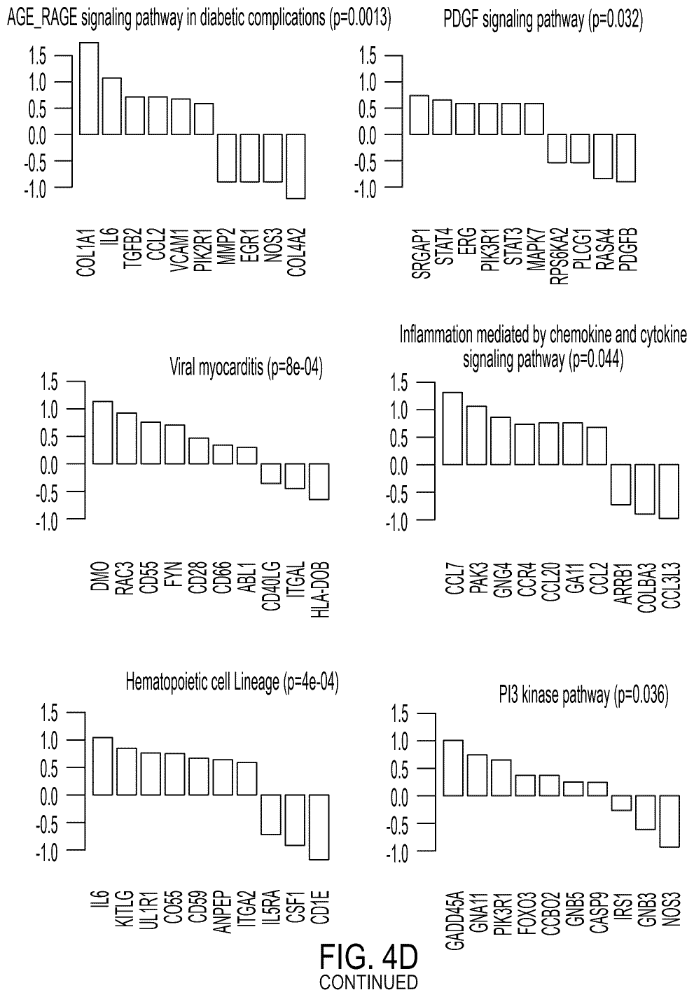

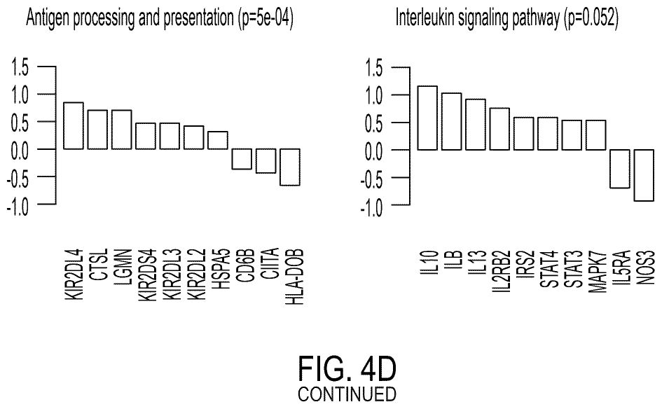

In parallel experiments, the inventors carried out RNA-seq analysis of the same healthy PBMCs stimulated with the same atherosclerotic (n=10) or healthy donor plasma (n=6). Principal component analysis (PCA) of all mapped genes and of corresponding CyTOF data (FIG. 3) clearly separated PBMCs stimulated with patient plasma from those stimulated with plasma of healthy donors. DESeq identified 4,823 differentially expressed genes (DEGs, FDR 5%) out of 39,129. Of the DEGs, 2,377 were up- and 2,446 down-regulated, suggesting a substantial transcriptional reprogramming in PBMCs exposed to atherosclerotic compared to healthy plasma (FIG. 3). Gene set enrichment, including Gene Ontology (GO) analysis using Enrichr (http://amp.pharm.mssm.edu/Enrichr), supports the involvement of protein transport, cell-cell signaling, and increased chemotaxis (FIG. 3), along with several biological processes relevant for innate immune responses in the observed response. For the DEGs, KEGG and PANTHER pathway enrichment analyses identified a series of up-and down-regulated signaling pathways implicated in the immune response to atherosclerotic plasma, including inflammation mediated by chemokine and cytokine, interleukin signaling pathways, PDGF signaling, antigen processing and presentation, PI3, and MAPK signaling pathways (FIG. 4).

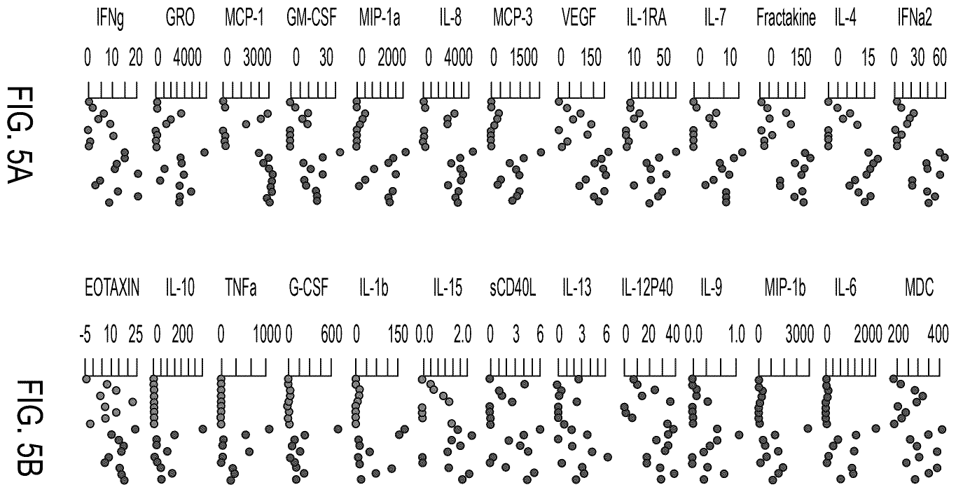

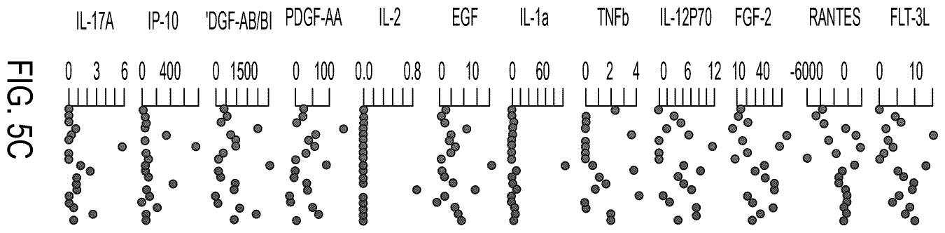

Together, these results show that plasma of atherosclerotic patients induces an inflammatory response in monocytes and CD1c⁺ DCs. To further evaluate the downstream consequences of these signaling and transcriptional pathways, the inventors performed a multiplexed profiling of soluble factors released by PBMCs following exposure to patient plasma and identified increases in several inflammatory cytokines (FIG. 5). Among others, the inventors identified cytokines that are causally implicated in atherosclerotic disease, such as IL-6, and chemokines such as MCP-1 and fractalkine, which are involved in monocyte/macrophage and T cell recruitment to tissues, potentially relating to the T cell infiltration that the inventors identified in human atherosclerotic tissue.

### Example 3

### Integrative Analysis of Cell Type-Intracellular Signaling Pair and Gene Expression

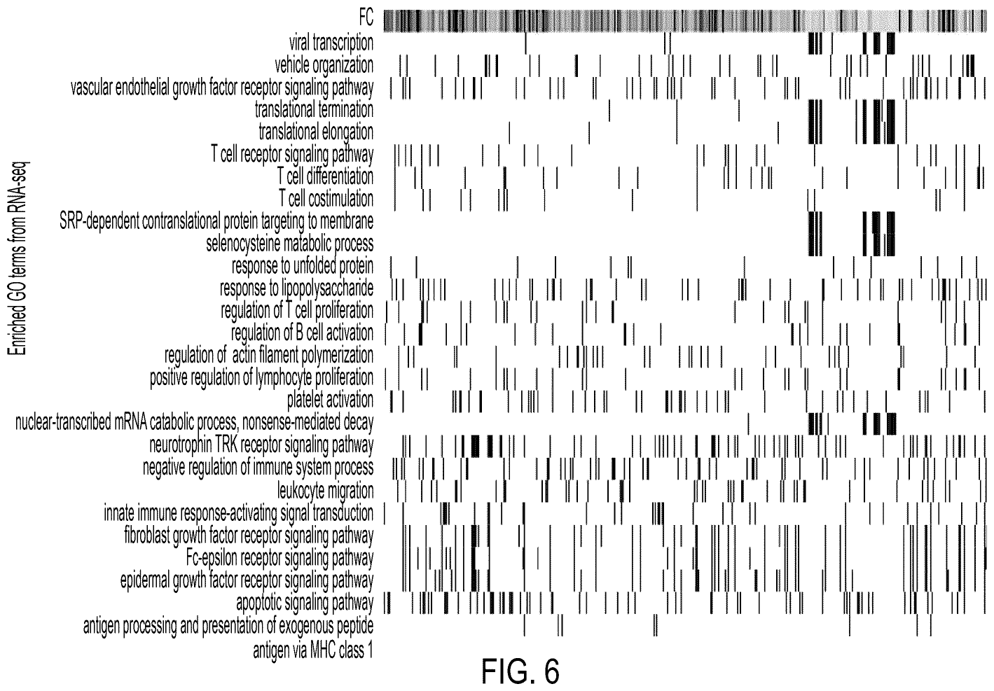

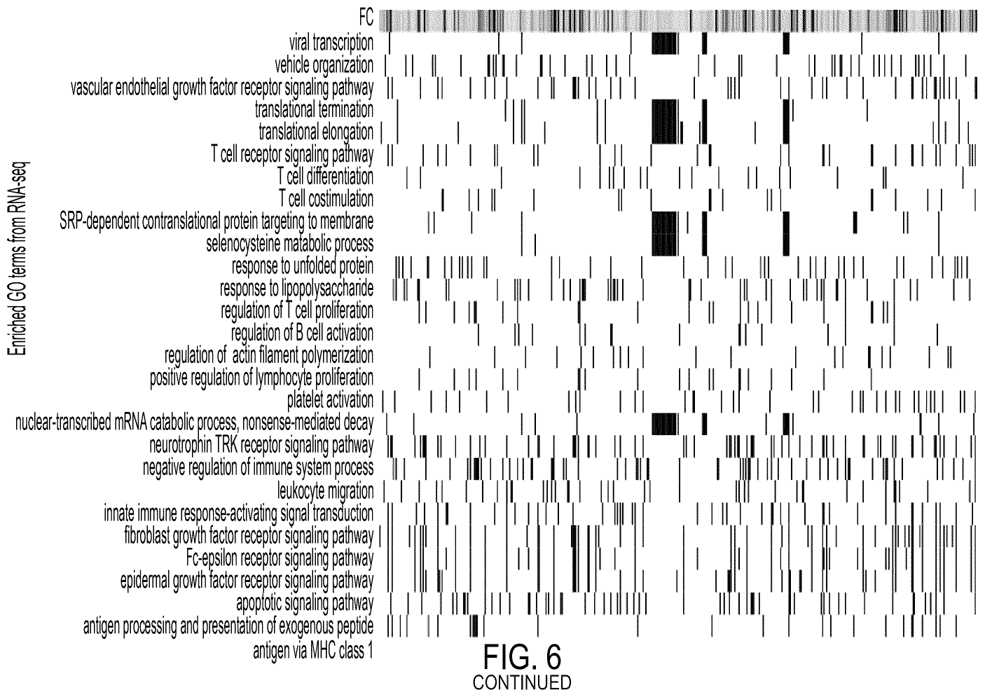

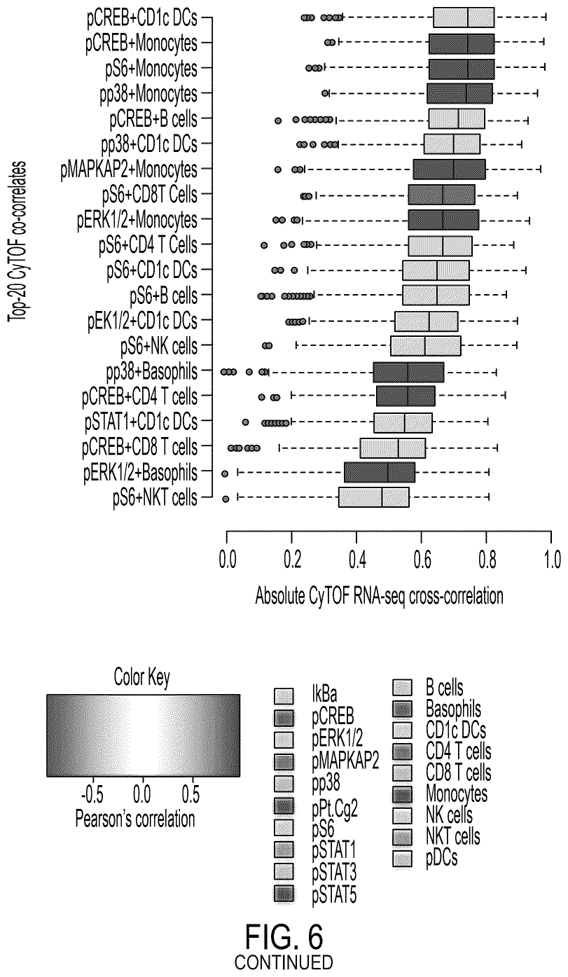

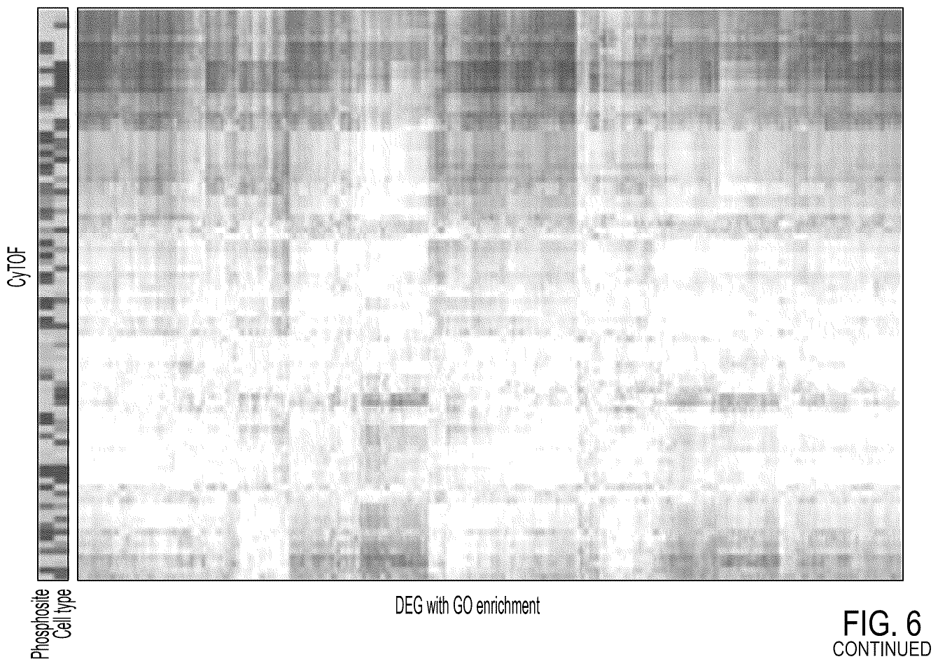

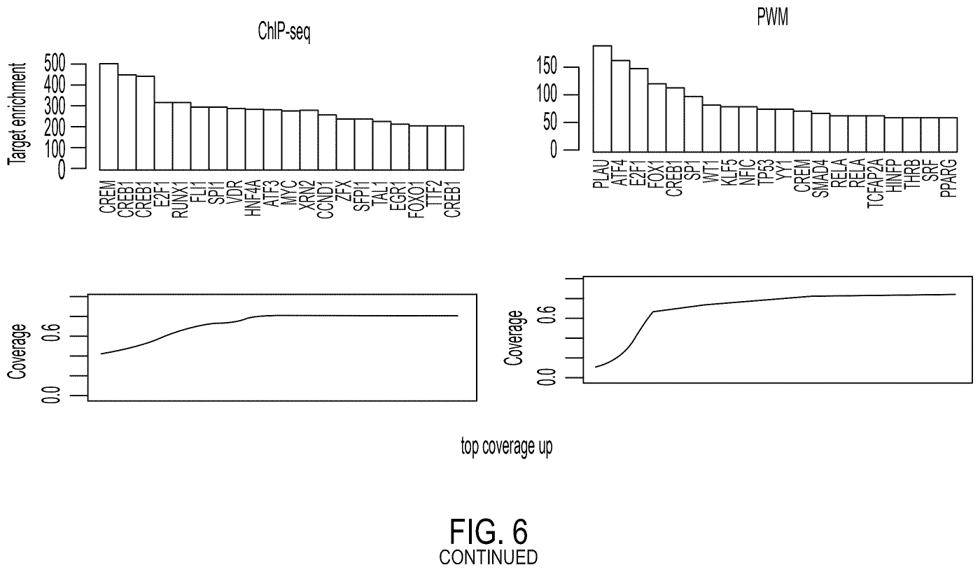

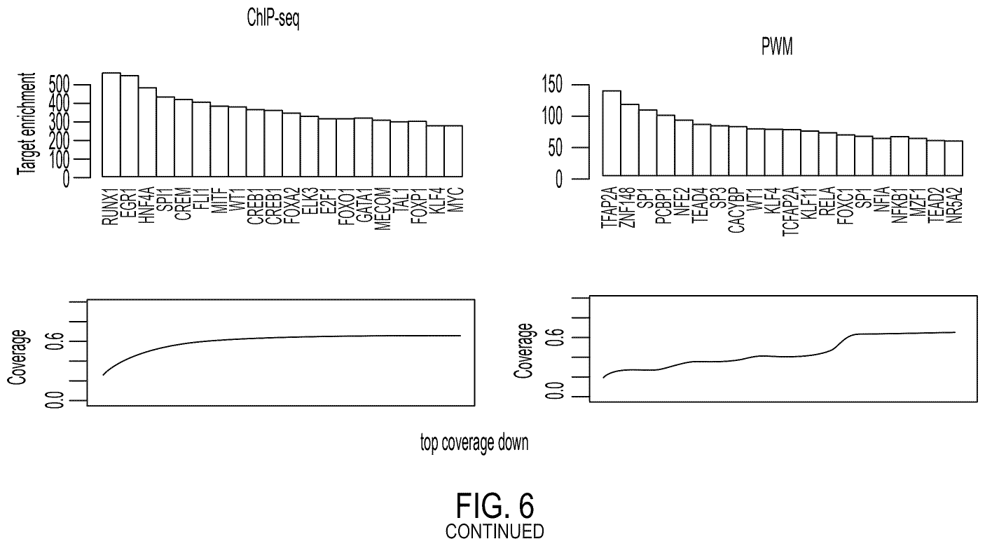

To discover possible regulatory relationships between cell type-specific signaling pathways and gene expression, the inventors integrated the analysis of GO enriched gene expression with identified pairs of cell type and phosphoprotein activity (FIG. 6). Filtered cross-correlations of mass cytometry and gene expression data identified CREB phosphorylation in dendritic cells and monocytes and S6 phosphorylation in monocytes as top correlates (FIG. 6). Analysis of DEGs against ChiP-seq libraries—ENCODE and ChEA Consensus from ChIP-X and ChEA 2015—and sequence motif predictions (TRANSFAC and JASPAR PWMs) identified CREB1, CREM—a CREB family member—and E2F1—a transcription factor (TF) that cooperates in the regulation of CREB signaling—as likely TFs explaining the expression of genes identified in PBMCs in response to patient plasma. Considering that CREB is activated in response to the phosphorylation of multiple kinases including S6, these data reveal that CREB phosphorylation, observed in monocytes and dendritic cells, is a critical signal contributing to the inflammatory transcriptional reprogramming triggered by atherosclerotic plasma.

Next the inventors used computational approaches to infer small molecules targeting the identified inflammatory transcriptional response in monocytes and DCs. RNA-seq of PBMCs stimulated with atherosclerotic plasma was first used to derive a signature of 4823 differentially expressed genes (DEGs) using DESeq R package with normalization based on size factors at a false discovery rate (FDR) of 0.05. Next, the inventors derived a sub-network of 711 DEGs associated with inflammatory response (GO:0006954). Identified signatures were compared to large scale gene expression data using the L1000CDS search engine that comprises 389,031 perturbation experiments, covering sixty-two cell lines and 3,924 small molecules, calculated from LINCS L1000 dataset using the characteristic direction method. By querying the input gene lists the inventors identified candidate small molecules predicted to reverse the input gene sets for each comparison. With this method, the therapeutic prediction for drug-gene set pair was based on the hypothesis that a small molecule that has an opposing effect on gene expression to that of PBMCs stimulated with atherosclerotic plasma would be more likely to interfere with the inflammatory response.

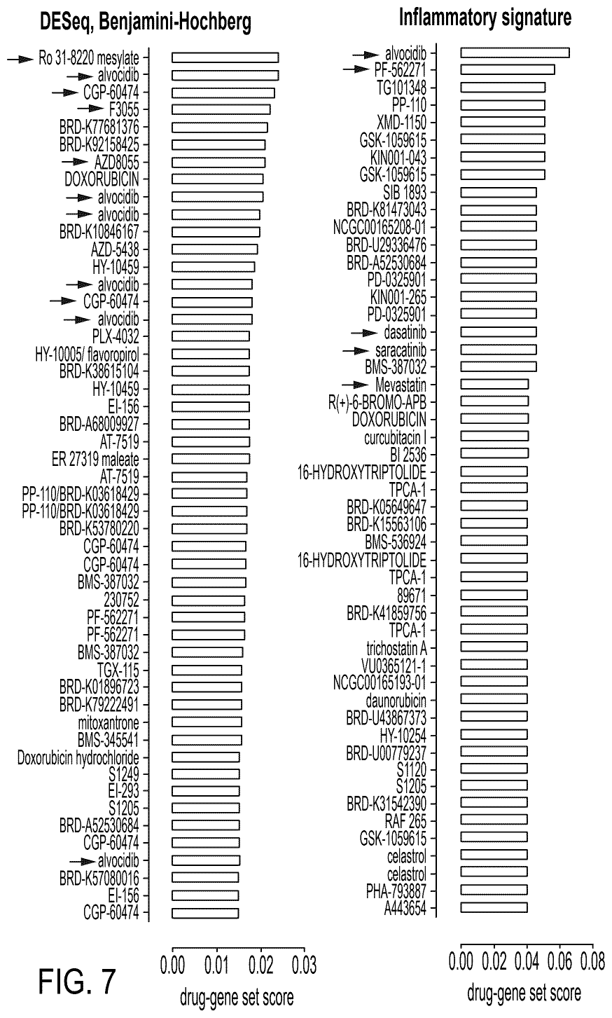

This approach identified several small molecules predicted to reverse the input inflammatory signatures seen in PBMCs stimulated with atherosclerotic plasma (FIG. 7). The input gene set of DESeq genes identified a set of small molecules that included the highest-scoring small molecule Ro 31-8220 Mesylate, a PKC inhibitor, alvocidib, a flavonoid alkaloid CDK9 kinase inhibitor, CGP-60474, a cyclin-dependent kinase (cdk) inhibitor, F3055, potent CDK (cyclin-dependent kinase), and a CDC25 phosphatase family inhibitor, and the mTOR inhibitor AZD8055. These compounds were selected for further validation. Among the small molecules identified using the subnetwork of 711 genes associated with inflammatory response (GO:0006954; Inflammatory signature), the inventors selected PF-562271, a FAK inhibitor, and dasatinib, and saracatinib. Mevastatin, the first discovered statin (a class of lipid lowering medications that are the pillar of CVD treatment) was also identified but with a lower drug-gene set score. Doxorubicin was also identified using both input gene sets but excluded from the list of drugs to be tested because of its low score and its cardiotoxicity.

### Example 4

### CyTOF Screening of Candidate Small Molecules to Test the Predicted Anti-Inflammatory Signaling

### Induced by Atherosclerotic Plasma

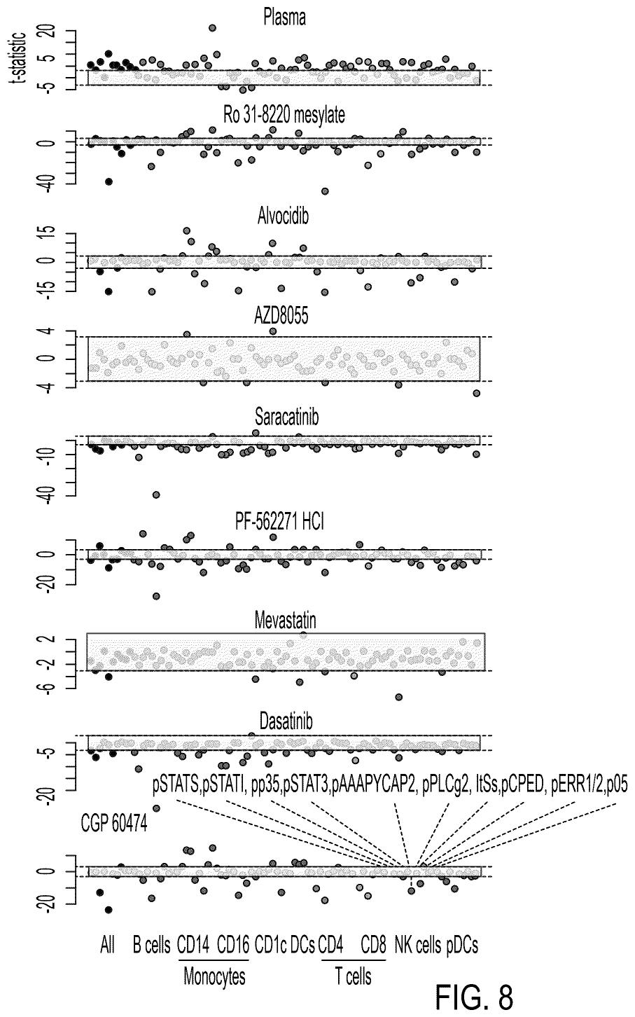

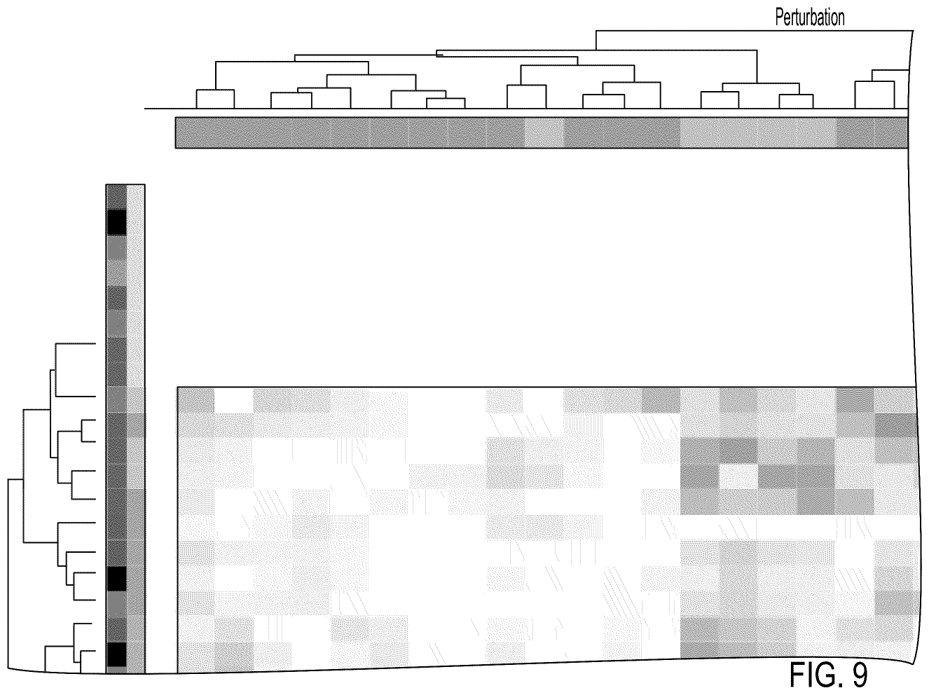

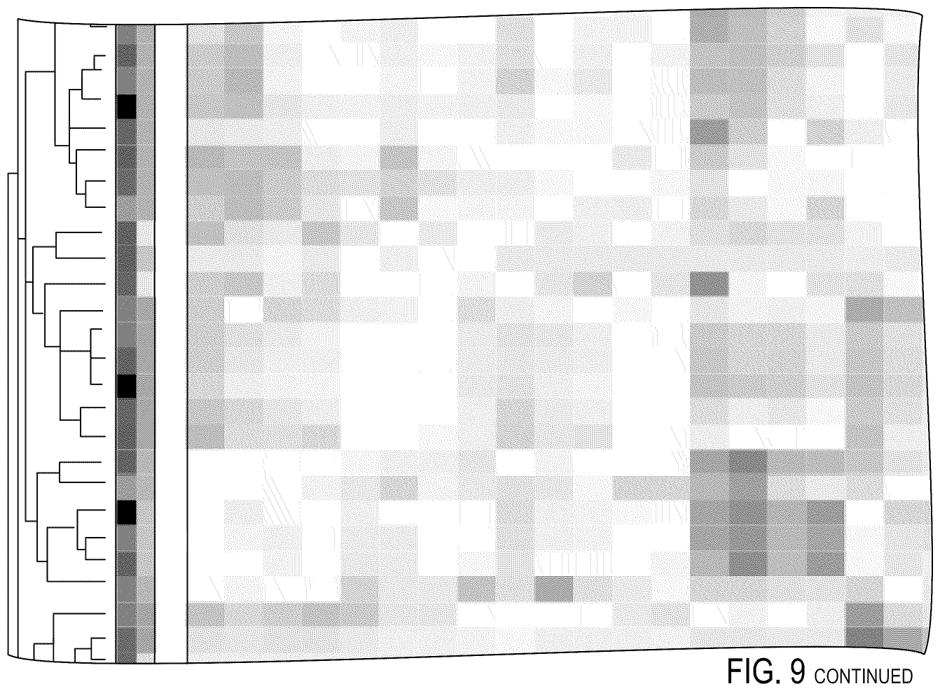

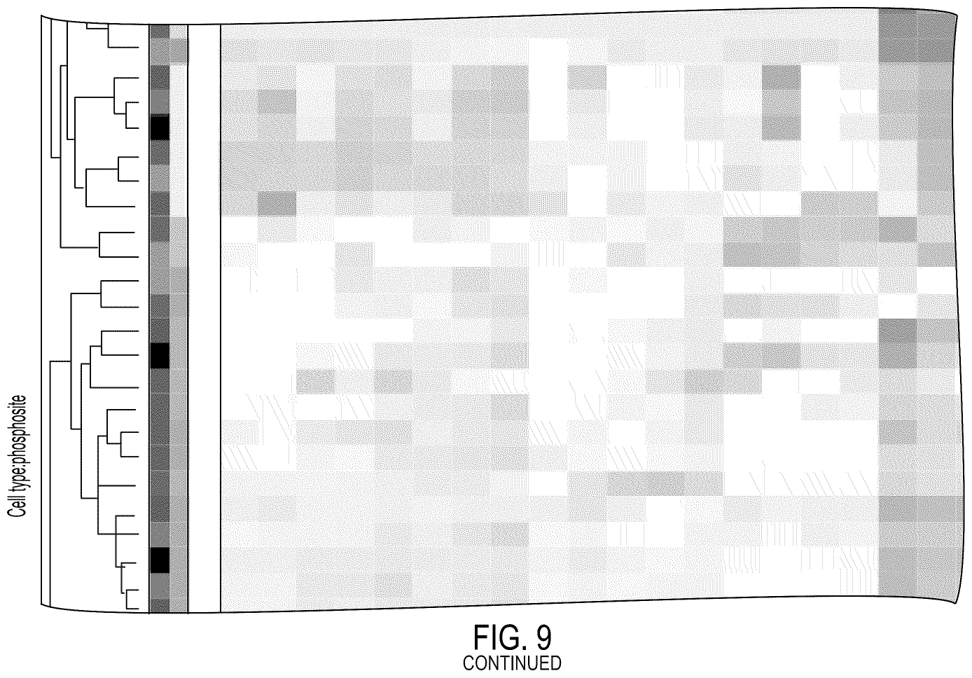

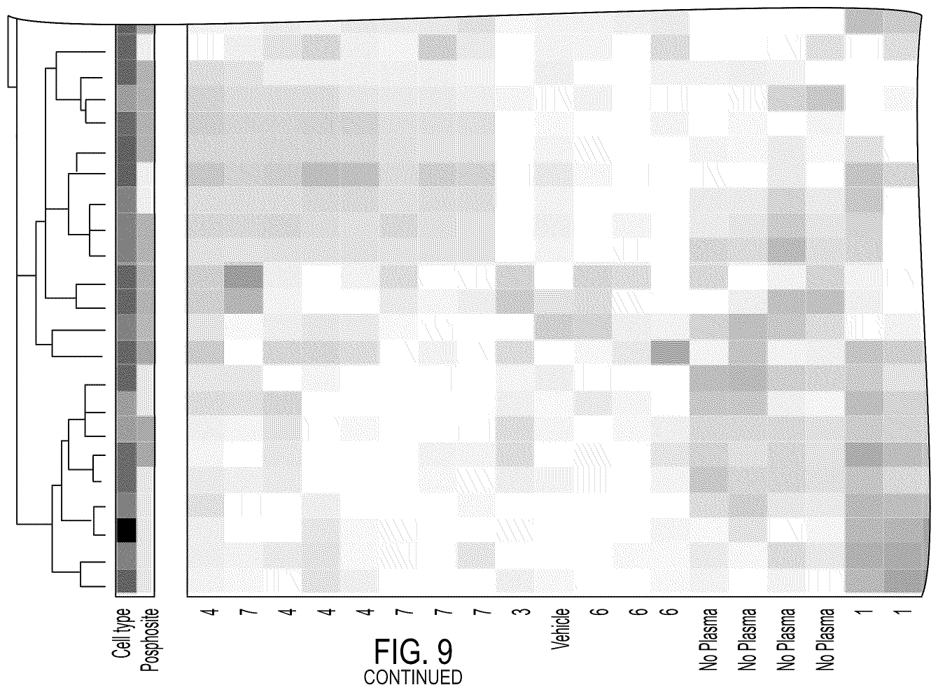

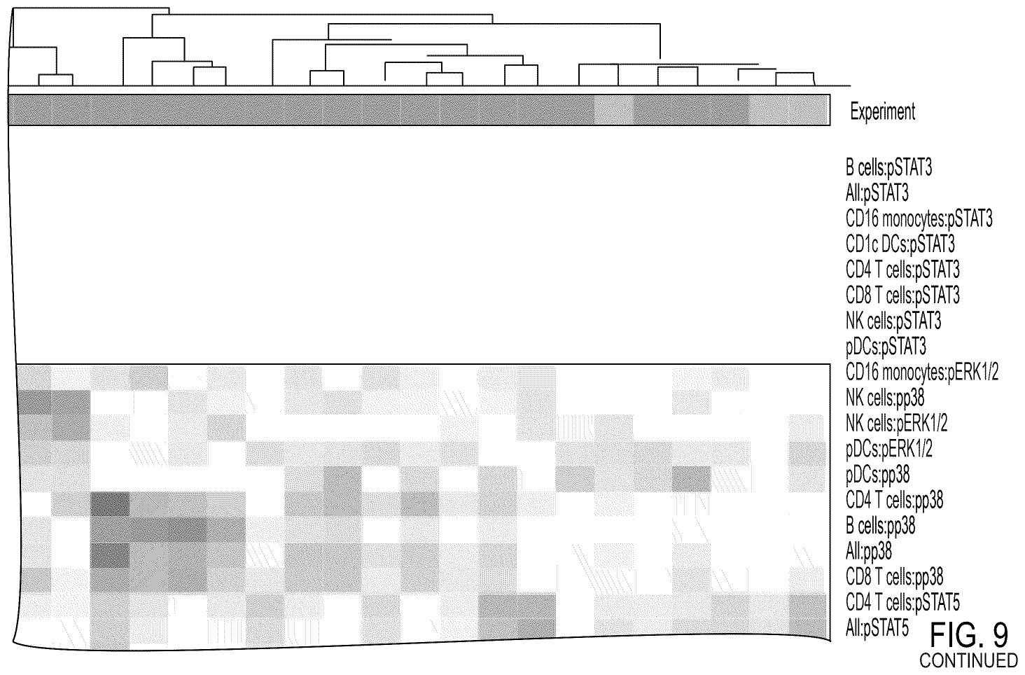

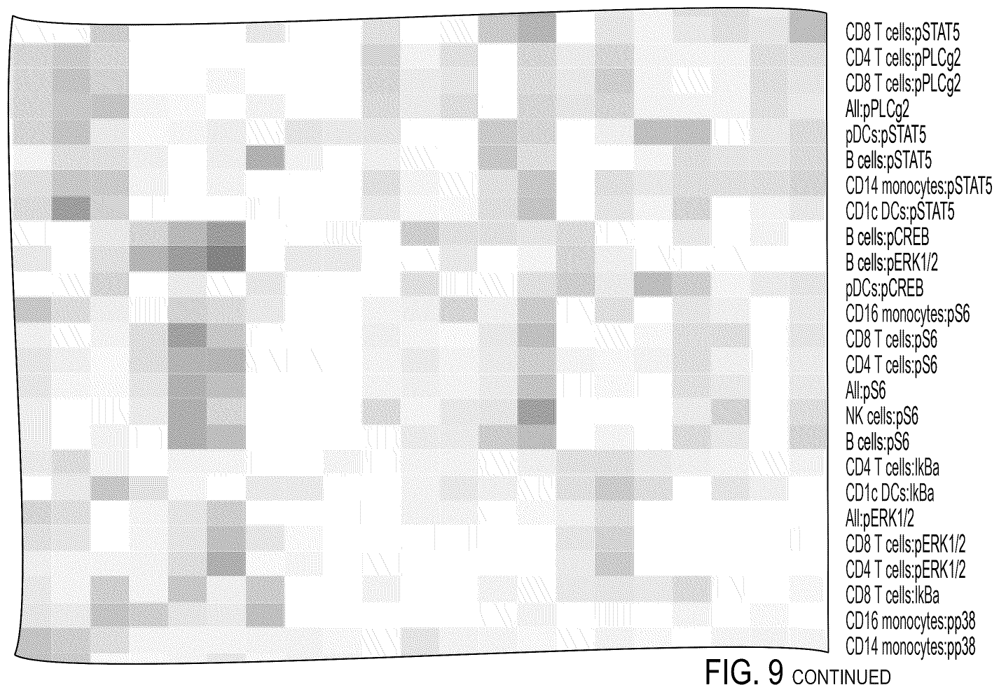

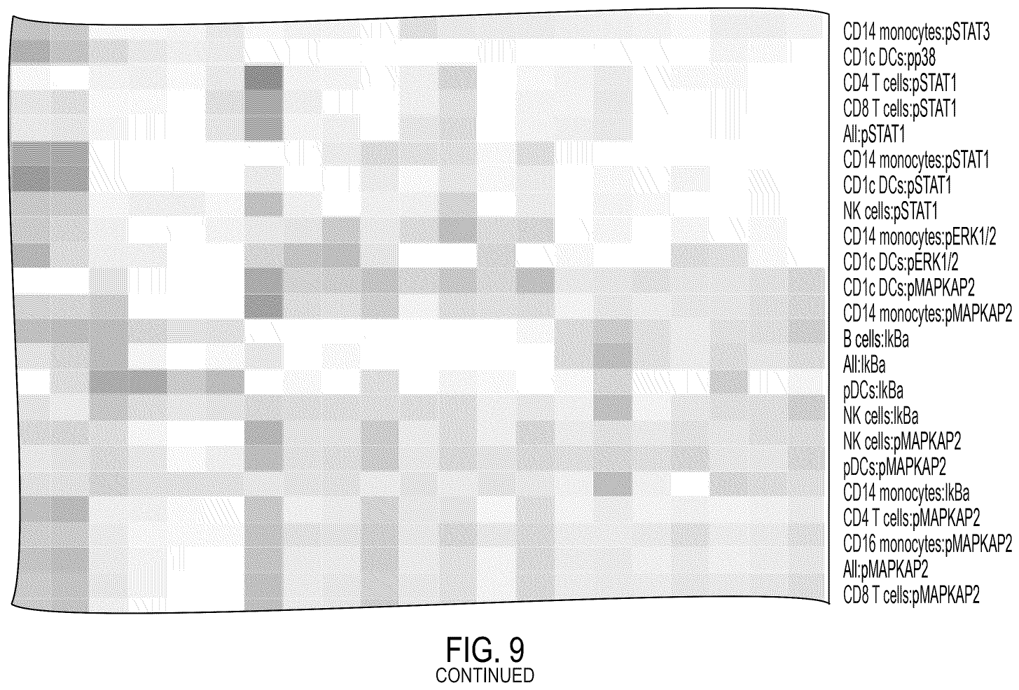

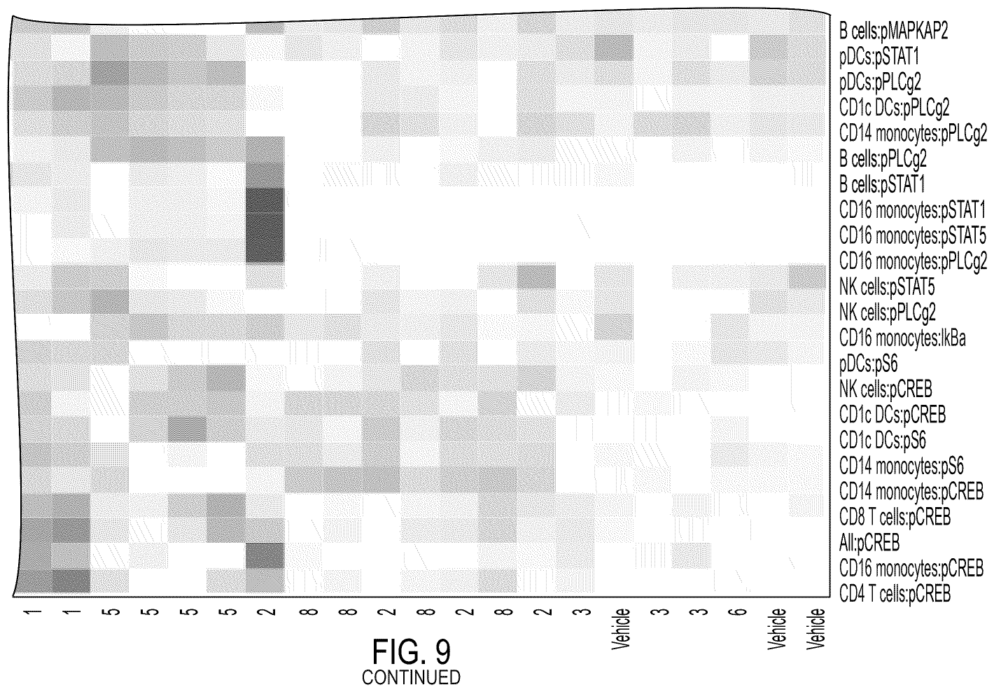

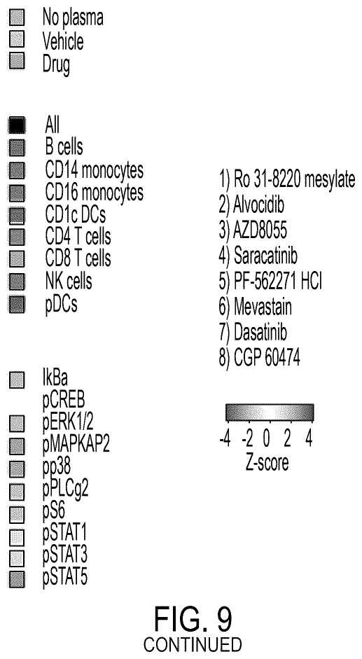

To test the efficacy of candidate small molecules in reversing immune response of PBMCs to atherosclerotic plasma, the inventors examined cell type-specific pharmacologic effects of selected candidate compounds. Using healthy PBMCs, the inventors assessed the activity of selected small molecules in the presence of atherosclerotic plasma stimulation that followed 30 minutes of pretreatment with each candidate compounds. Using CyTOF mass-cytometry, the inventors measured the specific inhibition of kinase phosphorylation (i.e., pCREB, pp38, pMAPKA2P, pS6, pERK1/2) in each cell type at the single cell level for each drug (FIGS. 8 and 9).

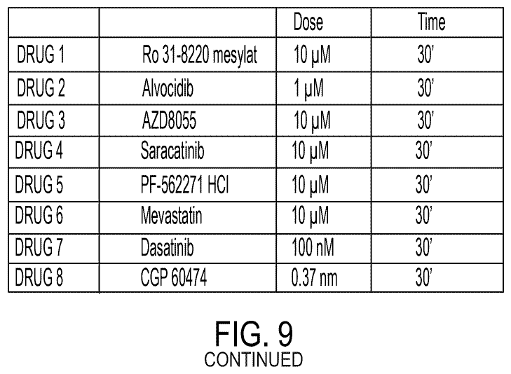

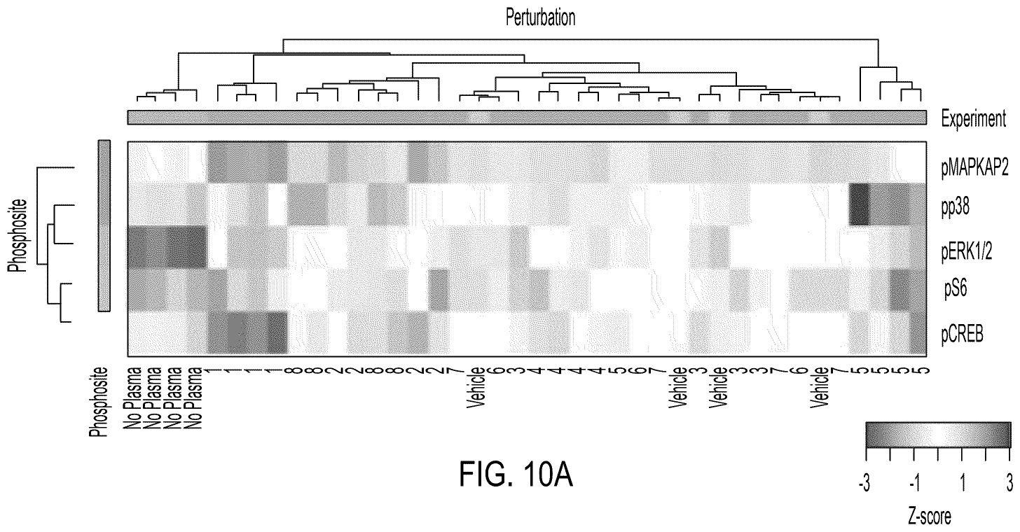

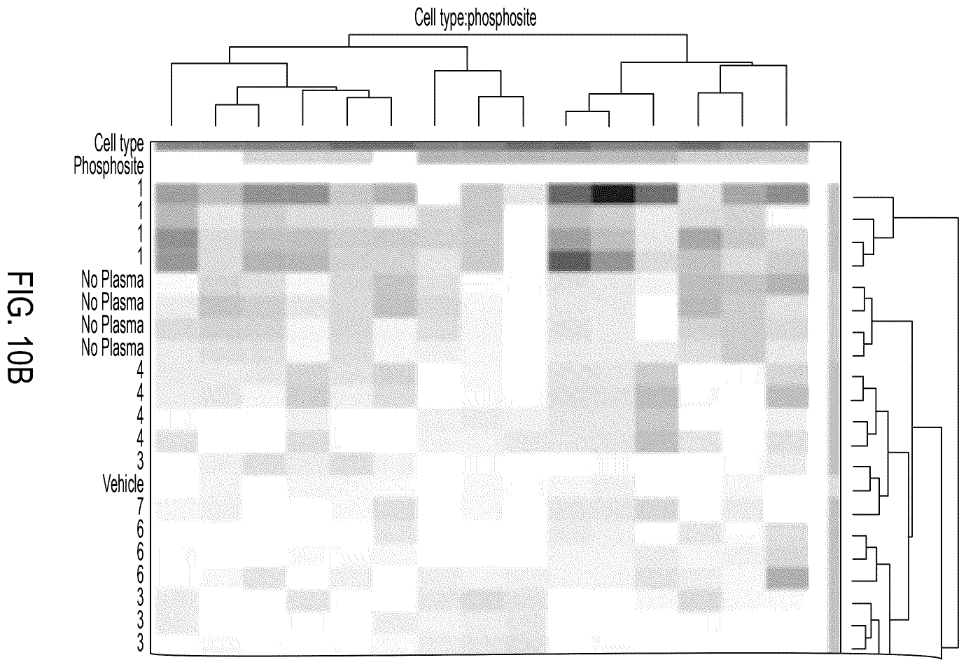

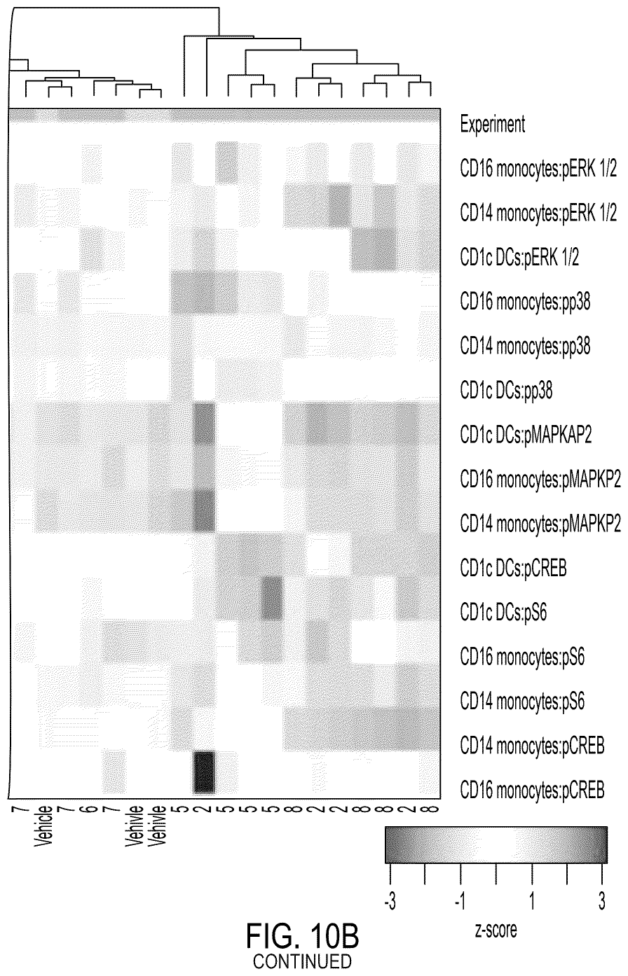

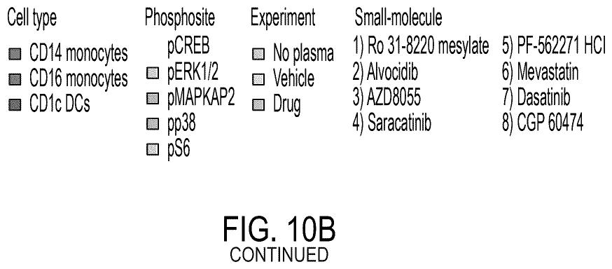

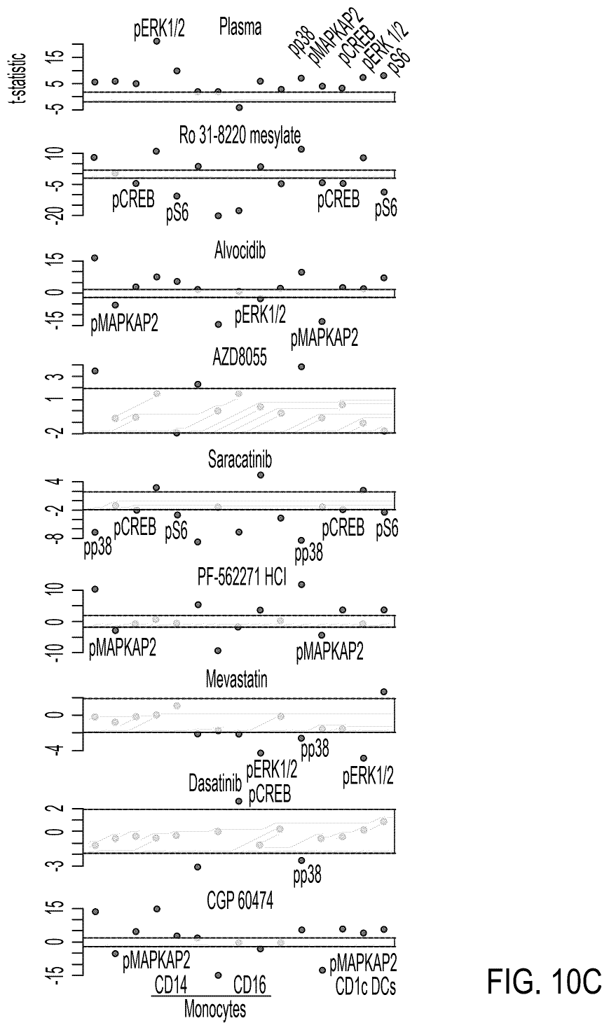

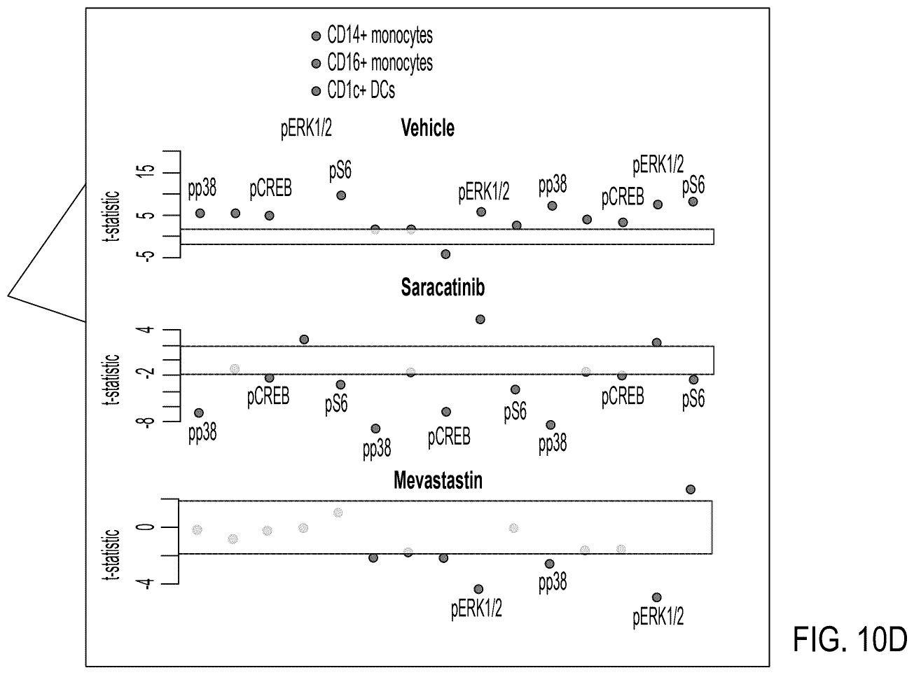

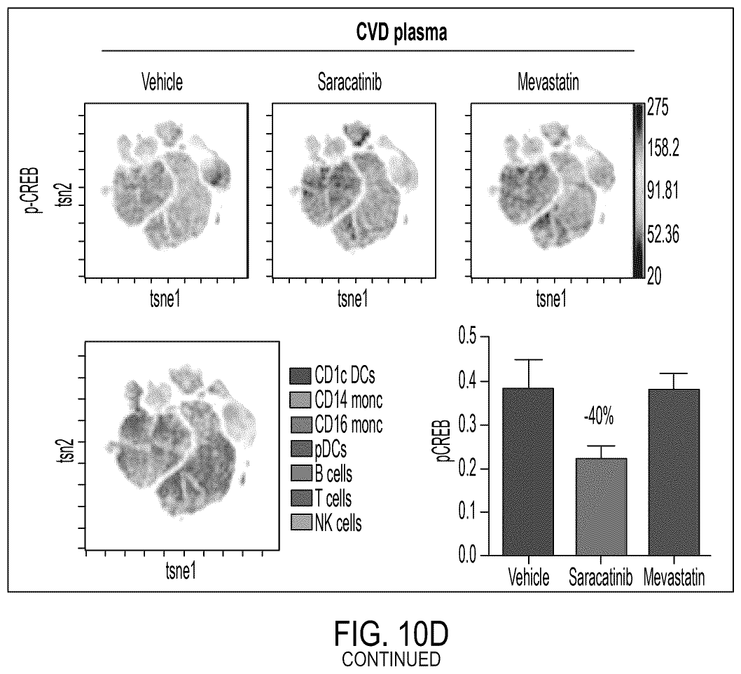

Among all tested compounds, R0 31-8220 Mesylate and Saracatinib induced a significant inhibition of most specific kinase phosphorylation activated by atherosclerosis plasma with cell-specificity for monocytes and CD1c⁺ DC (FIG. 10).

Specifically, Ro 31-8220 Mesylate induced a significant inhibition of CREB, S6 in CD14⁺ and CD16⁺ monocytes and in CD1c⁺ DC, and of MAPKA2P in CD16⁺ monocytes and in CD1c+ DC, while p38 and ERK1/2 phosphorylation in response to atherosclerosis plasma was not inhibited in these cells. Saracatinib produced a stronger inhibition based on the reduced phosphorylation of p38, CREB and S6 in both CD14⁺ and CD16⁺ monocytes, of MAPKA2P, CREB and S6 in cDC1⁺ DCs. Other tested compounds, including Mevastatin, did not affect the phosphorylation of major kinases (e.g., CREB and S6) implicated in the inflammatory transcriptional reprogramming activated by atherosclerotic plasma. For example, Mevastatin inhibited the phosphorylation of ERK1/2 in CD16⁺ monocytes and CD1c⁺ DC and of p38 in CD1c⁺ DC but did not fully reverse the inflammatory signature activated by atherosclerotic plasma.

### Example 5

### The Anti-Inflammatory Efficacy of the Saracatinib Directly in Circulating Inflammatory Cells From Patients With Carotid and Coronary Artery Disease

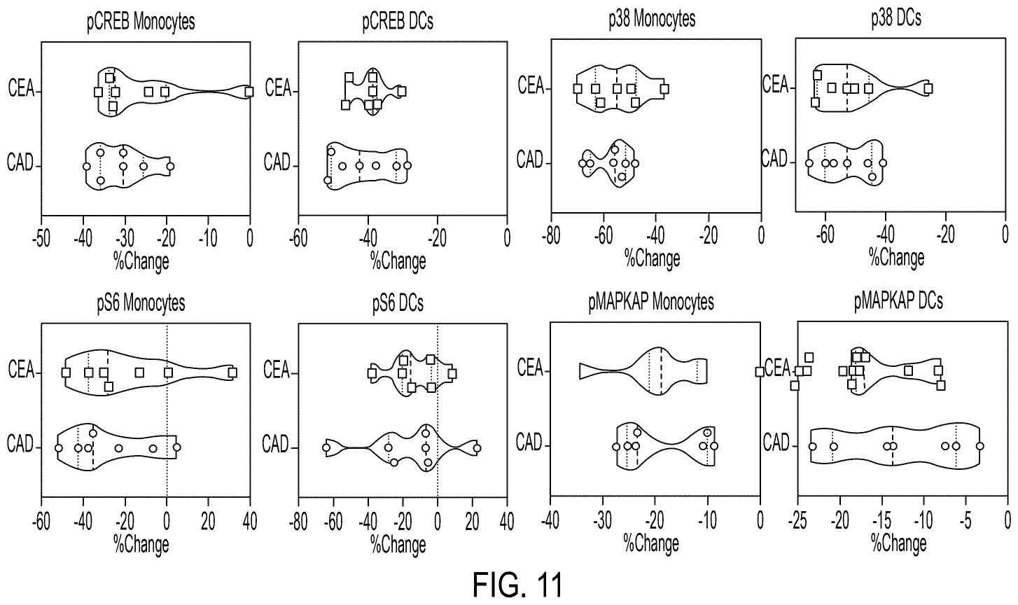

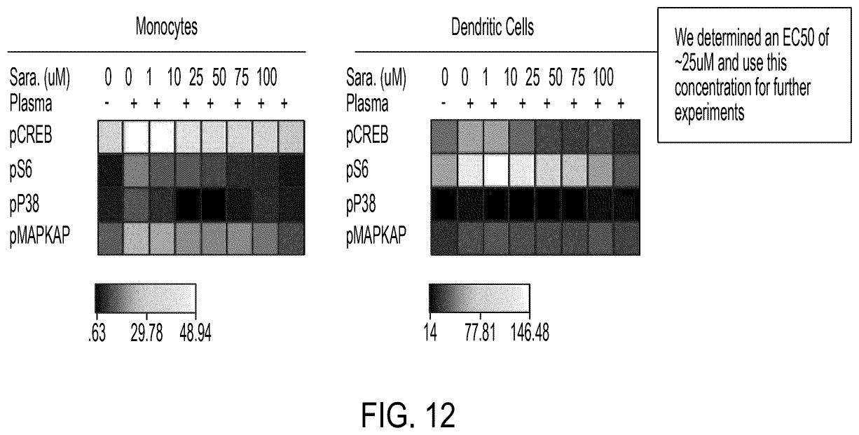

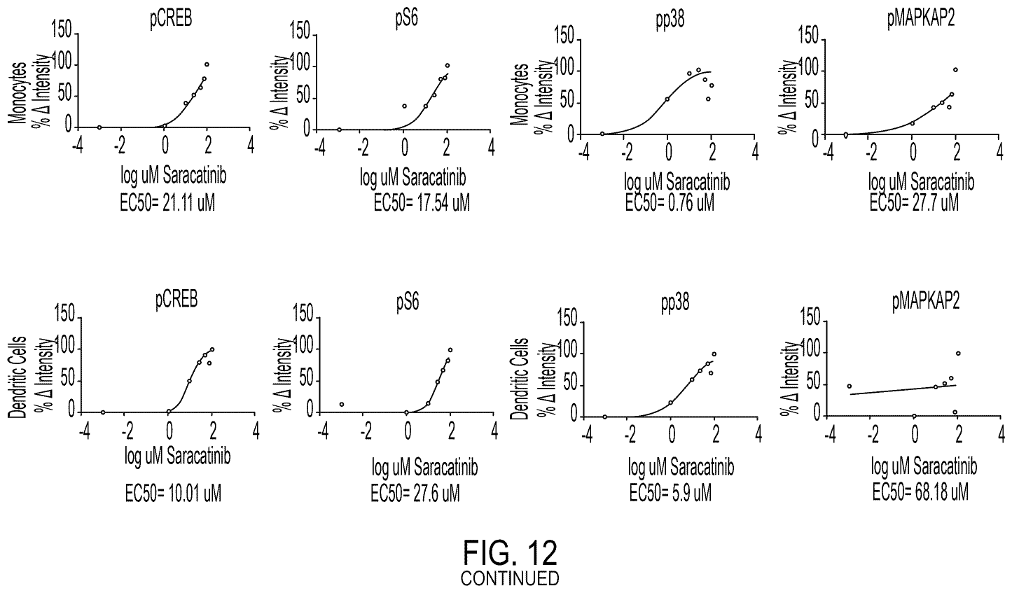

First, the inventors investigated the anti-inflammatory efficacy of saracatinib in PBMCs from patients with carotid (CEA) and coronary (CAD) atherosclerosis. Specifically, using CyTOF the inventors tested saracatinib for its efficacy to inhibit the phosphorylation of intracellular kinases in PBMCs of patients with carotid (CEA) and coronary artery disease (CAD) incubated with autologous vs. healthy donor plasma (FIG. 11).

### Example 6

### Effect of Saracatinib on Atherosclerosis Progression In Vivo

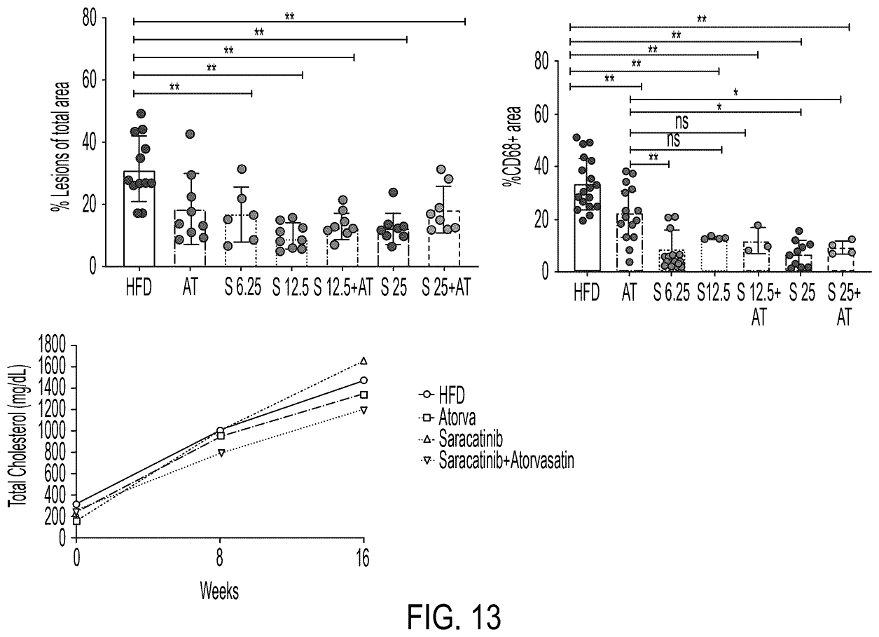

Apolipoprotein E-deficient (Apoe-/-, 6-weeks old, Jackson Lab) were used to measure the effect of the candidate drug on atherosclerotic plaque size and composition in vivo. Mice were randomized into four treatment groups: (1) western diet (WD) only (positive controls) (2) WD+oral atorvastatin (standard-of-care; 10 mg/kg/day) (3) WD+oral saracatinib (6.25 or 12.5 or 25 mg/kg/day); and (4) WD+oral atorvastatin+oral saracatinib ( 10/12.5 or 10/25 mg/kg/day). After 16 weeks of treatment, mice were euthanized. Plaque inflammation and burden were measured by histology. Effects on the arterial wall gene expression were measure by RNA sequencing. For RNAseq analysis, aortic tissue from each group was harvested and placed immediately in RNA Later (Ambion). For histological analysis, mice were perfused with 4% phosphate-buffered formaldehyde. For en face staining, aortas were opened longitudinally, pinned out flat on a silicon surface with the intima exposed and stained with Oil-Red-O for neutral lipids. Plaque burden was measured with Image Pro plus. For analysis of plaque composition (i.e. infiltration of inflammatory cell), the upper part of the heart and aorta was excised from mice of each group and embedded in OCT to generate aortic root sections for immunohistochemistry with antibodies against CD68 (monocyte/macrophages) and CD45 (pan-leukocytes). Saracatinib significantly reduced atherosclerotic plaque inflammation (CD68+ cells) and plaque burden/lesional area, and this effect was not related to changes in cholesterol levels (FIG. 13).

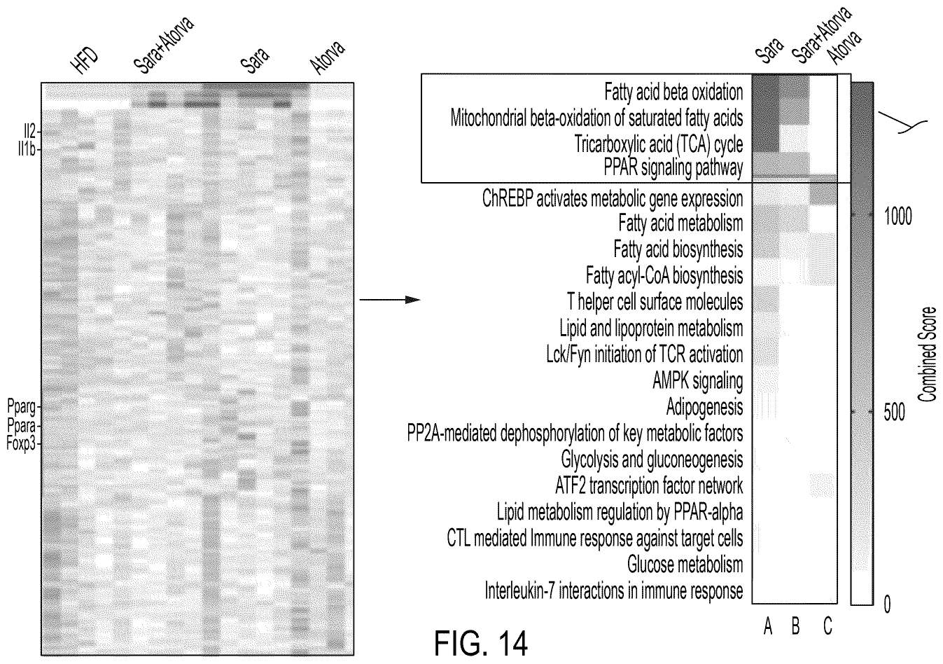

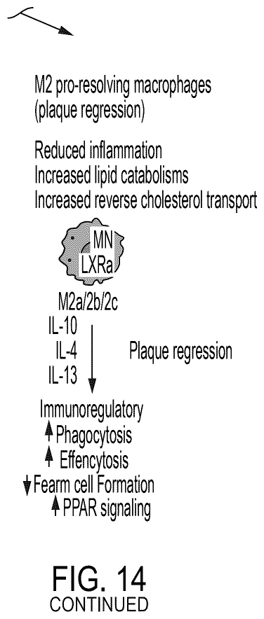

RNA sequencing analysis (FIG. 14) revealed the upregulation of genes involved in fatty acid oxidation, TCA cycles, and PPAR signaling in saracatinib treated mice. These pathways are upregulated in M2 pro-resolving macrophages that drive plaque regression. Fox-p3 a marker of T regs, also associated with regression, was also upregulated.

### Example 7

### Measuring Drug Efficacy In Vivo With Translational Pre-Clinical Imaging

To test the efficacy of saracatinib by non-invasive in vivo imaging directly translatable to humans, the inventors used a validated and reproducible pre-clinical rabbit model of atherosclerosis that develops complex atherosclerotic lesions, which are comparable to human plaque complexity. Atherosclerosis was induced in New Zealand White (NZW) rabbits by a combination of a high-fat diet (HFD) and 2 balloon endothelial denudations. Four months after diet initiation, all animals underwent baseline imaging. Immediately afterwards, animals were randomized into 1) positive controls (n=10) kept on high fat diet (HFD); 2) HFD+oral statins treatment group (n=10, 3 mg/Kg/day of oral atorvastatin); 3) HFD+saracatinib oral treatment group (n=10, 4 mg/Kg/day); and 4) HFD+saracatinib+atorvastatin oral treatment group (n=10, 4/3 mg/Kg/day). All groups were imaged at 3 months after treatment initiation to assess progression in the control group (1) and efficacy of: oral statins (2); saracatinib (3); and of statins+saracatinib (4) on halting progression or inducing regression.

¹⁸F-FDG, one of the most common PET tracers, is a glucose analog, which accumulates in metabolically active cells. ¹⁸F-FDG uptake (measured as SUV mean and maximum) correlates significantly and consistently with plaque macrophage content in both atherosclerotic rabbits and patients. In addition, ¹⁸F-FDG signal correlates with several circulating inflammatory biomarkers and expression levels of several genes, including CD68 (monocyte/macrophages), that are associated with atherosclerotic plaque inflammation, as well as with clinical risk factors or risk scores. The reliability of ¹⁸F-FDG PET and MRI of plaque burden has also been studied in several vascular territories in humans (carotid and femoral arteries, thoracic and abdominal aorta). Importantly, the rabbit aorta has approximately the same size of the human carotid, making these two vascular sites extremely comparable when using in vivo non-invasive imaging modalities. Immunohistochemistry: Abdominal aortas were embedded in paraffin for validation with conventional immunohistochemistry. RAM-11 (marker of macrophages in rabbits, equivalent to CD68 in mice) staining was used to quantify plaque macrophage content1, while ORO staining was used to quantify plaque lipid content.

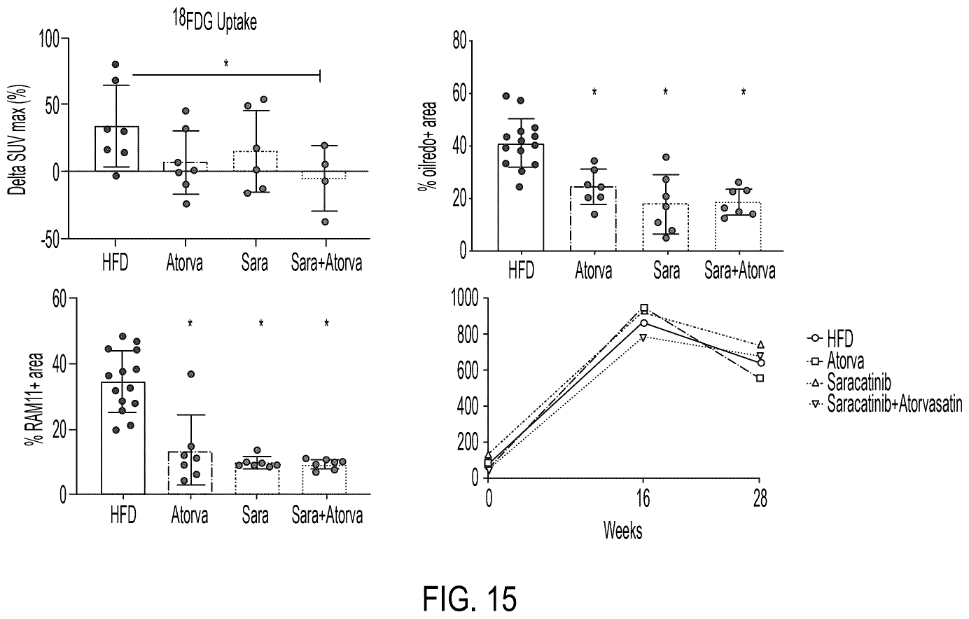

The inventors discovered that ¹⁸F-FDG-uptake was reduced by saracatinib alone or in combination with atorvastatin (FIG. 15). Histological analysis of rabbit plaques showed that plaque inflammation was reduced (as indicated by the PET results) and lipid content was also reduced. This result is in line with the gene expression result in mice showing that saracatinib increases M2 and cholesterol removal from existing plaques. The inventors found that anti-atherosclerotic effect on existing plaques was independent from lipid lowering.

### Example 8

### Identification of 5 Different Drugs That Show Anti-Inflammatory Effect Against the Effect of Atherosclerotic Plasma on Inflammatory Cells

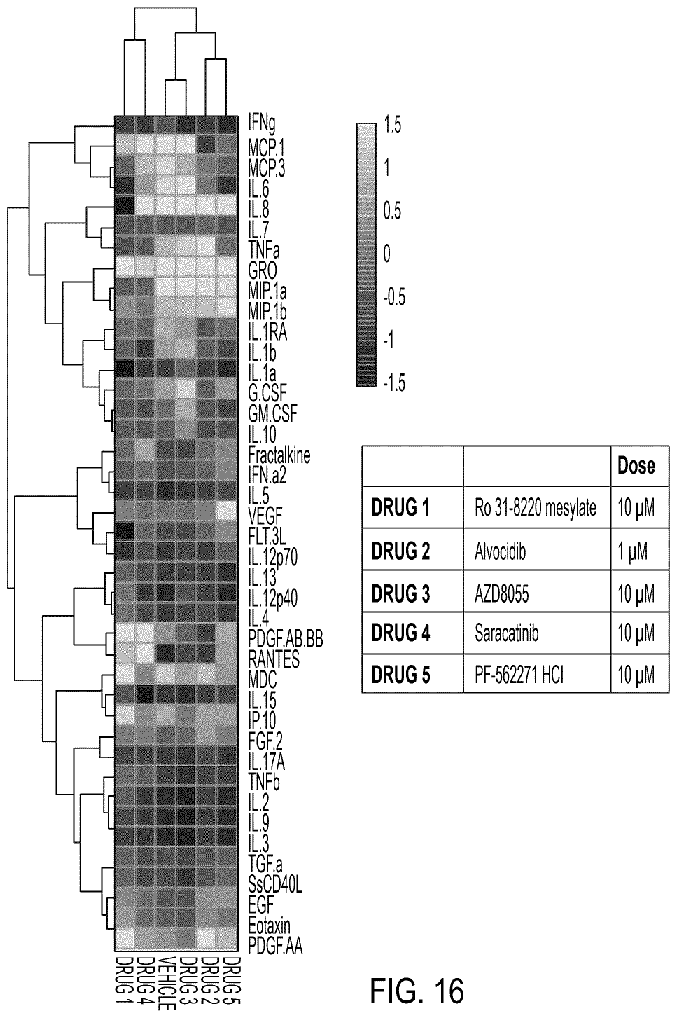

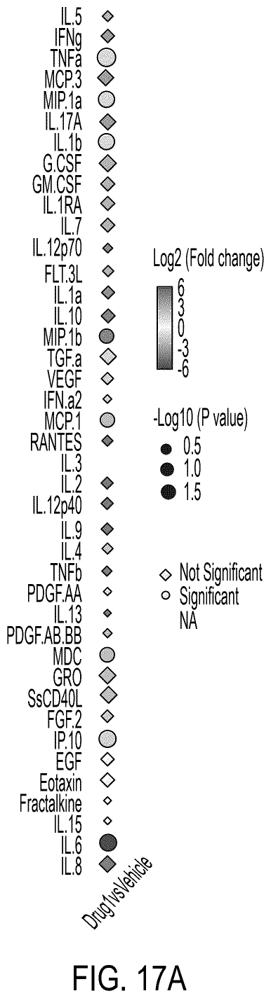

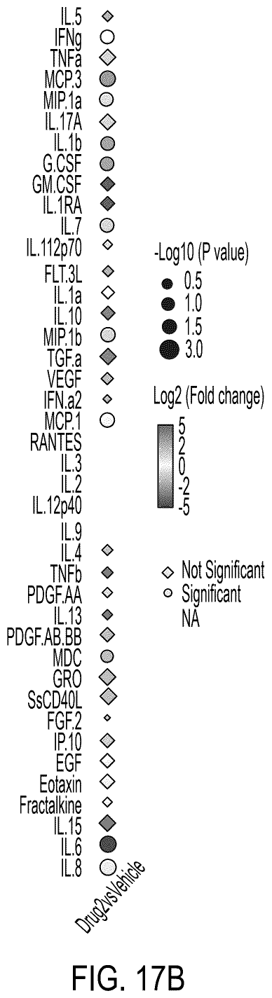

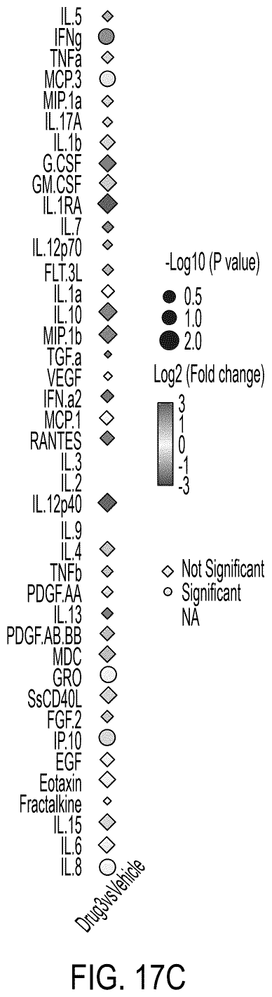

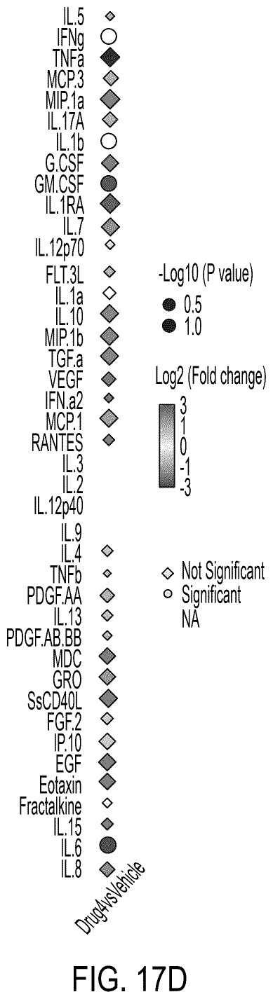

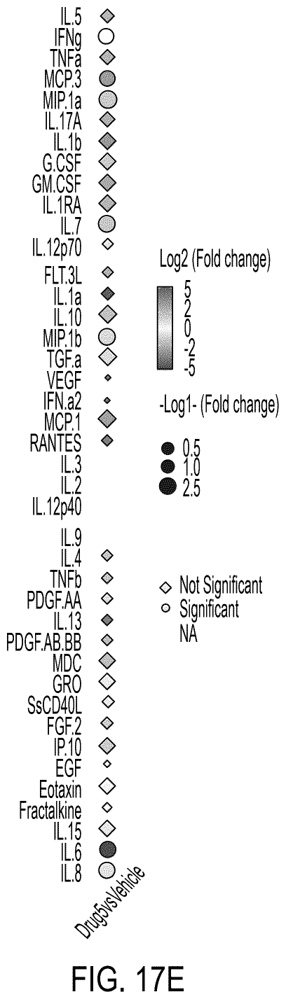

The inventors tested the effects of five drugs against inflammation induced in PMBCs by atherosclerotic plasma. All five drugs showed reduction in cytokine expression by PMBCs when compared to control (vehicle), with different levels of reduction in various cytokines, including, for example, IFNg, MCP.1, MCP.3, IL6, IL8, TNFa, MIP.1a, MIP.1b, IL 1Ra, IL 1b, MDC, IP.10 (FIGS. 16 and 17).

1. A method for treating cerebrovascular or cardiovascular disease in a subject in need thereof, the method comprising administering a compound selected from the group consisting of: Ro 31-8220 mesylate; Alvocidib; AZD8055; Saracatinib; and PF-562271 HCl.

2. The method of claim 1, wherein said administered compound is saracatinib.

3. The method of claim 1, wherein said administered compound is Ro 31-8220 mesylate.

4. The method of claim 1, wherein said administered compound is Alvocidib.

5. The method of claim 1, wherein said administered compound is AZD8055.

6. The method of claim 1, wherein said administered compound is PF-562271 HCl.

7. The method of claim 1, further comprising administering a drug selected from the group consisting of: statins, PCSK9 inhibitors, and ezetimibe.

8. The method of claim 7, wherein the statin is selected from the group consisting of: atorvastatin, mevastatin, fluvastatin, lovastatin, pitavastatin, pravastatin, rosuvastatin, and simvastatin.

9. The method of claim 7, wherein the PCSK9 inhibitor is selected from the group consisting of evolocumab and alirocumab.

10. A method for reducing inflammation associated with cardiovascular disease in a subject in need thereof, the method comprising administering a compound selected from the group consisting of: Ro 31-8220 mesylate; Alvocidib; AZD8055; Saracatinib; and PF-562271 HCl.

11. The method of claim 10, wherein said administered compound is saracatinib.

12. The method of claim 10, wherein said administered compound is Ro 31-8220 mesylate.

13. The method of claim 10, wherein said administered compound is Alvocidib.

14. The method of claim 10, wherein said administered compound is AZD8055.

15. The method of claim 10, wherein said administered compound is PF-562271 HCl

16. The method of claims 10, further comprising administering a drug selected from the group consisting of: statins, PCSK9 inhibitors, and ezetimibe.

17. The method of claim 16, wherein the statin is selected from the group consisting of: atorvastatin, mevastatin, fluvastatin, lovastatin, pitavastatin, pravastatin, rosuvastatin, and simvastatin.

18. The method of claim 16, wherein the PCSK9 inhibitor is selected from the group consisting of evolocumab and alirocumab.

19. The method of claim 7 further comprising administering ezetimibe.

20. The method of claim 16 further comprising administering ezetimibe.
